(19) United States
(12) Patent Application Publication
(10) Pub. No.: US 2024/0301450 A1
(43) Pub. Date: Sep. 12, 2024

(54) NOVEL METHOD FOR PREPARING POLY(3-HYDROXYBUTYRATE-CO-HYDROXYBUTYRATE)

(71) Applicant: CJ CHEILJEDANG CORPORATION, Seoul (KR)

(72) Inventors: Yae Seul PARK, Seoul (KR); Hyang CHOI, Seoul (KR); Dong-Eun CHANG, Seoul (KR); Ji Sun LEE, Seoul (KR); Imsang LEE, Seoul (KR)

(73) Assignee: CJ CHEILJEDANG CORPORATION, Seoul (KR)

(21) Appl. No.: 18/180,507

(22) Filed: Mar. 8, 2023

(51) Int. Cl. C12P 7/625; C12N 1/20; C12N 9/04; C12N 9/10; C12N 9/88; C12N 15/70

(52) U.S. Cl. CPC C12P 7/625; C12N 1/20; C12N 9/0006; C12N 9/1029; C12N 9/13; C12N 9/88; C12N 15/70; C12R 2001/19; C12Y 101/01035; C12Y 101/01157; C12Y 203/01009; C12Y 208/03001; C12Y 402/01017; C12Y 402/0112

(57) ABSTRACT

The present disclosure relates to a novel method for preparing poly(3-hydroxybutyrate-co-4-hydroxybutyrate), a microorganism using the biosynthetic pathway of poly(3-hydroxybutyrate-co-4-hydroxybutyrate) of the present disclosure, a composition for producing poly(3-hydroxybutyrate-co-4-hydroxybutyrate), and a method for regulating the 4-hydroxybutyrate content of poly(3-hydroxybutyrate-co-4-hydroxybutyrate).

### TECHNICAL FIELD

The present disclosure relates to a novel method for preparing poly(3-hydroxybutyrate-co-4-hydroxybutyrate), a microorganism using the biosynthetic pathway of poly(3-hydroxybutyrate-co-4-hydroxybutyrate) of the present disclosure, a composition for producing poly(3-hydroxybutyrate-co-4-hydroxybutyrate), and a method for regulating the 4-hydroxybutyrate content of poly(3-hydroxybutyrate-co-4-hydroxybutyrate).

### BACKGROUND ART

Most plastics used worldwide are produced based on petrochemicals, and since they do not decompose naturally, such plastics are becoming the main cause of environmental pollution. While research is being actively conducted on biomass-based bioplastics as a substitute for petrochemical-based plastics, in particular, research on the production of polyhydroxyalkanoates (PHA), which are biodegradable biomaterials produced using microorganisms, is attracting attention.

There are more than 150 types of monomers capable of constituting polyhydroxyalkanoates, and the structure and physical properties of biodegradable polymers vary depending on the composition of the constituent monomers. In particular, production technologies for various types of biodegradable polymer are being developed as a result of the wider range of commercialization allowed by the physical properties of polymers that vary in this way.

In this regard, 3-hydroxybutyrate homopolymer (poly(3-hydroxybutyrate), P(3HB)), which has been most widely studied among polyhydroxyalkanoate-based polymers in microorganisms, has a high degree of crystallinity, making it brittle and hard, and decomposes near its melting point, and thus, it has limited processability. Meanwhile, a novel copolymer formed by adding a 4-hydroxybutyrate monomer to a 3-hydroxybutyrate homopolymer has different physical properties from the 3-hydroxybutyrate homopolymer, and as a result, polymers are produced that can be used for various purposes.

In general, 3-hydroxybutyrate and 4-hydroxybutyrate are each produced via acetyl-CoA and the tricarboxylic acid (TCA) cycle. Specifically, 3-hydroxybutyrate is produced from acetyl-CoA through acetoacetyl-CoA and 3-hydroxybutyryl-CoA, and 4-hydroxybutyrate is produced from succinyl-CoA, succinic acid semialdehyde, 4-hydroxybutyric acid, and 4-hydroxybutyryl-CoA in the TCA cycle.

Depending on the 4-hydroxybutyrate content, the properties of the poly(3-hydroxybutyrate-co-4-hydroxybutyrate) change from hard crystalline to elastic rubber. Therefore, it is necessary to control the 4-hydroxybutyrate content in order to produce poly(3-hydroxybutyrate-co-4-hydroxybutyrate) having flexible physical and mechanical properties. However, the 4-hydroxybutyrate production through the TCA cycle is closely related to strain growth, and thus, it is difficult to control its content.

Therefore, research for producing poly(3-hydroxybutyrate-co-4-hydroxybutyrate) having various 4-hydroxybutyrate contents is still required.

### DISCLOSURE

### Technical Problem

It is one object of the present disclosure to provide a method for preparing poly(3-hydroxybutyrate-co-4-hydroxybutyrate).

Specifically, the present disclosure provides a method for preparing poly(3-hydroxybutyrate-co-4-hydroxybutyrate), including:
(a) converting crotonyl-CoA into 3-hydroxybutyrate using enoyl-CoA hydratase and/or converting crotonyl-CoA into 4-hydroxybutyrate using 4-hydroxybutyryl-CoA dehydratase; and
(b) preparing poly(3-hydroxybutyrate-co-4-hydroxybutyrate) by polymerization of 3-hydroxybutyrate and 4-hydroxybutyrate using PHA synthase

In any one embodiment, the preparation method of poly(3-hydroxybutyrate-co-4-hydroxybutyrate) of the present disclosure may further include the following (i) to (iii), or (iv) step prior to step (a) above:
(i) converting acetyl-CoA into acetoacetyl-CoA using CoA acetyltransferase;
(ii) converting acetoacetyl-CoA into 3-hydroxybutyryl-CoA using 3-hydroxybutyryl-CoA dehydrogenase;
(iii) converting 3-hydroxybutyryl-CoA into crotonyl-CoA using 3HB-CoA dehydratase; or
(iv) converting crotonate into crotonyl-CoA using propionyl CoA-transferase.

In any one embodiment, in the preparation method of poly(3-hydroxybutyrate-co-4-hydroxybutyrate) of the present disclosure, the activity of enoyl-CoA hydratase and/or the activity of 4-hydroxybutyryl-CoA dehydratase may be regulated.

In any one of the above-described embodiments, in the preparation method of poly(3-hydroxybutyrate-co-4-hydroxybutyrate) of the present disclosure, the activity of enoyl-CoA hydratase activity may be weakened, and/or the activity of 4-hydroxybutyryl-CoA dehydratase activity may be enhanced.

In any one embodiment, in the preparation method of poly(3-hydroxybutyrate-co-4-hydroxybutyrate) of the present disclosure, the poly(3-hydroxybutyrate-co-4-hydroxybutyrate) may have a 4-hydroxybutyrate content of 0.1% to 60%.

It is another object of the present disclosure to provide a microorganism having poly(3-hydroxybutyrate-co-4-hydroxybutyrate) productivity.

Specifically, the present disclosure provides a microorganism having poly(3-hydroxybutyrate-co-4-hydroxybutyrate) productivity, including a gene encoding enoyl-CoA hydratase, a gene encoding 4-hydroxybutyryl-CoA dehydratase, and a gene encoding PHA synthase.

In any one embodiment, in the microorganism having poly(3-hydroxybutyrate-co-4-hydroxybutyrate) productivity, the microorganism may belong to the genus Escherichia.

In any one embodiment, in the microorganism having poly(3-hydroxybutyrate-co-4-hydroxybutyrate) productivity, the microorganism of the genus Escherichia may be Escherichia coli.

In any one embodiment, in the microorganism having poly(3-hydroxybutyrate-co-4-hydroxybutyrate) productivity, the microorganism may further include: (i′) a gene encoding acetyl-CoA acetyltransferase, a gene encoding 3-hydroxybutyryl-CoA dehydrogenase, and a gene encoding 3HB-CoA dehydratase; or (ii′) a gene encoding propionyl CoA-transferase.

In any one embodiment, in the microorganism having poly(3-hydroxybutyrate-co-4-hydroxybutyrate) productivity, the activity of enoyl-CoA hydratase activity may be weakened, and/or the activity of 4-hydroxybutyryl-CoA dehydratase activity may be enhanced.

In any one embodiment, in the microorganism having poly(3-hydroxybutyrate-co-4-hydroxybutyrate) productivity, the poly(3-hydroxybutyrate-co-4-hydroxybutyrate) may have a 4-hydroxybutyrate content of 0.1% to 60%.

It is still another object of the present disclosure to provide a composition for producing poly(3-hydroxybutyrate-co-4-hydroxybutyrate).

Specifically, the present disclosure provides a composition for producing poly(3-hydroxybutyrate-co-4-hydroxybutyrate), including: enoyl-CoA hydratase, 4-hydroxybutyryl-CoA dehydratase, and PHA synthase; a microorganism expressing the same; or a culture of the microorganism.

In any one embodiment, in the composition for producing poly(3-hydroxybutyrate-co-4-hydroxybutyrate), the composition may further include: (i″) acetyl-CoA acetyltransferase, 3-hydroxybutyryl-CoA dehydrogenase, and 3HB-CoA dehydratase; a microorganism expressing the same; or a culture of the microorganism; or (ii″) propionyl CoA-transferase; a microorganism expressing the same; or a culture of the microorganism.

In any one embodiment, in the composition for producing poly(3-hydroxybutyrate-co-4-hydroxybutyrate), the poly(3-hydroxybutyrate-co-4-hydroxybutyrate) may have a 4-hydroxybutyrate content of 0.1% to 60%.

It is yet another object of the present disclosure to provide a method for regulating the 4-hydroxybutyrate content of poly(3-hydroxybutyrate-co-4-hydroxybutyrate).

Specifically, the present disclosure provides a method for regulating the 4-hydroxybutyrate content of poly(3-hydroxybutyrate-co-4-hydroxybutyrate), including: (a) converting crotonyl-CoA into 3-hydroxybutyrate using enoyl-CoA hydratase and/or converting crotonyl-CoA into 4-hydroxybutyrate using 4-hydroxybutyryl-CoA dehydratase; and (b) preparing poly(3-hydroxybutyrate-co-4-hydroxybutyrate) by polymerization of 3-hydroxybutyrate and 4-hydroxybutyrate using PHA synthase; and weakening the activity of enoyl-CoA hydratase and/or enhancing the activity of 4-hydroxybutyryl-CoA dehydratase, prior to step (a) above.

In any one embodiment, in the method for regulating the 4-hydroxybutyrate content of poly(3-hydroxybutyrate-co-4-hydroxybutyrate), the poly(3-hydroxybutyrate-co-4-hydroxybutyrate) may have a 4-hydroxybutyrate content of 0.1% to 60%.

In any one embodiment, in the method for regulating the 4-hydroxybutyrate content of poly(3-hydroxybutyrate-co-4-hydroxybutyrate), the step of weakening the activity of enoyl-CoA hydratase may be performed by a method selected from the group consisting of reduction in intracellular copy number of a polynucleotide encoding a polypeptide, replacement of a promoter, genomic insertion of a gene encoding a polypeptide, and a combination thereof.

### Technical Solution

The present disclosure will be described in detail. Meanwhile, each description and embodiment disclosed herein can be applied to other descriptions and embodiments, respectively. That is, all combinations of various elements disclosed herein fall within the scope of the present disclosure. Further, the scope of the present disclosure is not limited by the specific description described below. Additionally, a number of papers and patent documents have been cited throughout the present specification. The content of the cited papers and patent documents is incorporated herein by reference in their entirety and the level of technical field to which the present disclosure belongs and the contents of the present disclosure will be described more clearly.

One aspect of the present disclosure provides a method for preparing poly(3-hydroxybutyrate-co-4-hydroxybutyrate).

As used herein, the term “poly(3-hydroxybutyrate-co-4-hydroxybutyrate) (P(3HB-co-4HB))” is one of polyhydroxyalkanoates (PHA), and refers to a biodegradable polyester in which 3-hydroxybutyrate and 4-hydroxybutyrate are combined.

As used herein, the term “poly(3-hydroxybutyrate) (P(3HB))” is a compound belonging to polyesters as a polymer of 3-hydroxybutyrate. The poly(3-hydroxybutyrate) may be used interchangeably with poly-3-hydroxybutanoate (P3HA) and 3-hydroxybutyrate homopolymer.

As used herein, the term “poly(4-hydroxybutyrate) (P(4HB))” is a compound belonging to polyesters as a polymer of 4-hydroxybutyrate. The poly(4-hydroxybutyrate) may be used interchangeably with poly-4-hydroxybutanoate (P4HA) and 4-hydroxybutyrate homopolymer.

In one embodiment, the method for preparing poly(3-hydroxybutyrate-co-4-hydroxybutyrate) of the present disclosure may be carried out using microorganisms, and may be carried out by microbial culture.

Specifically, the method for preparing poly(3-hydroxybutyrate-co-4-hydroxybutyrate) of the present disclosure may include: (a) converting crotonyl-CoA into 3-hydroxybutyrate and/or 4-hydroxybutyrate; and
(b) preparing poly(3-hydroxybutyrate-co-4-hydroxybutyrate) by polymerization of 3-hydroxybutyrate and 4-hydroxybutyrate.

Each step may be performed sequentially or in situ in the same reaction system.

More specifically, the method for preparing poly(3-hydroxybutyrate-co-4-hydroxybutyrate) of the present disclosure may include: (a) converting crotonyl-CoA into 3-hydroxybutyrate using enoyl-CoA hydratase and/or converting crotonyl-CoA into 4-hydroxybutyrate using 4-hydroxybutyryl-CoA dehydratase; and
(b) preparing poly(3-hydroxybutyrate-co-4-hydroxybutyrate) by polymerization of 3-hydroxybutyrate and 4-hydroxybutyrate using PHA synthase.
   Each step above may be performed sequentially or in situ in the same reaction system.

As used herein the term “enoyl-CoA hydratase” refers to an enzyme that converts crotonyl-CoA into 3-hydroxybutyrate, specifically (R)-3-hydroxybutyrate.

The gene encoding the enoyl-CoA hydratase may be -phaJ derived from Aeromonas caviae, and may specifically consist of the nucleotide sequence of SEQ ID NO: 6. Specifically, any nucleotide sequence exhibiting a homology of 60% or more, 70% or more, preferably 80% or more, more preferably 90% or more, even more preferably 95% or more, and most preferably 99% or more with the sequence of SEQ ID NO: 6 may be included without limitation, as long as it is substantially capable of expressing a protein having enoyl-CoA hydratase activity.

As used herein, the term “4-hydroxybutyryl-CoA dehydratase” refers to an enzyme that converts crotonyl-CoA into 4-hydroxybutyrate.

The gene encoding the 4-hydroxybutyryl-CoA dehydratase may be 4hbd derived from Nitrosopumilus maritimus, Candidatus Nitrosopelagicus brevis, Candidatus Nitrosarchaeum limnium, or Thaumarchaeota archaeon, and may specifically consist of any one of the nucleotide sequences of SEQ ID NOS: 7 to 11. Specifically, any nucleotide sequence exhibiting a homology of 60% or more, 70% or more, preferably 80% or more, more preferably 90% or more, even more preferably 95% or more, and most preferably 99% or more with the sequence of SEQ ID NOS: 7 to 11 may be included without limitation, as long as it is substantially capable of expressing a protein having 4-hydroxybutyryl-CoA dehydratase activity.

As used herein, the term “PHA synthase” refers to an enzyme that catalyzes the polymerization of 3-hydroxybutyrate and 4-hydroxybutyrate to produce poly(3-hydroxybutyrate-co-4-hydroxybutyrate).

The gene encoding PHA synthase may be phaC derived from Ralstonia eutropha, Ralstonia solanacearum, Zoogloea ramigera, Delftia acidovorans, Bacillus megaterium, Cromobacterium sp. USM2, Rhodobacter sphaeroides, Chromobacterium sp. USM2, Azohydromonas australica, Azohydromonas lata, Acidovorax species, Burkholderia pseudomallei, Burkholderia vietnamiensis, or Burkholderia glumae, specifically Ralstonia eutropha, and may specifically consist of the nucleotide sequence of SEQ ID NO: 2. Specifically, any nucleotide sequence exhibiting a homology of 60% or more, 70% or more, preferably 80% or more, more preferably 90% or more, even more preferably 95% or more, and most preferably 99% or more with the sequence of SEQ ID NO: 2 may be included without limitation, as long as it is substantially capable of expressing a protein having PHA synthase activity.

The novel pathway to produce poly(3-hydroxybutyrate-co-4-hydroxybutyrate) from crotonyl-CoA of the present disclosure has been implemented for the first time by the present inventors.

Specifically, the preparation pathway of the novel poly(3-hydroxybutyrate-co-4-hydroxybutyrate) of the present disclosure may be referred to FIG. 1.

In one embodiment, the method for preparing poly(3-hydroxybutyrate-co-4-hydroxybutyrate) of the present disclosure may further include the following (i) to (iii), or (iv) step, prior to step (a) above, but is not limited thereto:
(i) converting acetyl-CoA into acetoacetyl-CoA using CoA acetyltransferase;
(ii) converting acetoacetyl-CoA into 3-hydroxybutyryl-CoA using 3-hydroxybutyryl-CoA dehydrogenase;
(iii) converting 3-hydroxybutyryl-CoA into crotonyl-CoA using 3HB-CoA dehydratase; or
(iv) converting crotonate into crotonyl-CoA using propionyl CoA-transferase.

Each step above may be performed sequentially or in situ in the same reaction system.

In step (iv) of the present disclosure, the crotonate may be added from the outside.

As used herein, the term “acetyl-CoA acetyltransferase” refers to an enzyme that converts acetyl-CoA into acetoacetyl-CoA.

The gene encoding acetyl-CoA acetyltransferase may be phaA derived from Ralstonia eutropha, and may specifically consist of the nucleotide sequence of SEQ ID NO: 1. Specifically, any nucleotide sequence exhibiting a homology of 60% or more, 70% or more, preferably 80% or more, more preferably 90% or more, even more preferably 95% or more, and most preferably 99% or more with the sequence of SEQ ID NO: 1 may be included without limitation, as long as it is substantially capable of expressing a protein having acetyl-CoA acetyltransferase activity.

As used herein, the term “3-hydroxybutyryl-CoA dehydrogenase” refers to an enzyme that converts acetoacetyl-CoA into 3-hydroxybutyryl-CoA.

The gene encoding 3-hydroxybutyryl-CoA dehydrogenase may be hbd derived from Clostridium acetobutylicum, and may specifically consist of the nucleotide sequence of SEQ ID NO: 4. Specifically, any nucleotide sequence exhibiting a homology of 60% or more, 70% or more, preferably 80% or more, more preferably 90% or more, even more preferably 95% or more, and most preferably 99% or more with the sequence of SEQ ID NO: 4 may be included without limitation, as long as it is substantially capable of expressing a protein having 3-hydroxybutyryl-CoA dehydrogenase activity.

As used herein, the term “3HB-CoA dehydratase” refers to an enzyme that converts 3-hydroxybutyryl-CoA, specifically (S)-3-hydroxybutyrate, into crotonyl-CoA. The 3HB-CoA dehydratase may be used interchangeably with enoyl-CoA hydratase.

The gene encoding 3HB-CoA dehydratase may be crt derived from Clostridium acetobutylicum, and may specifically consist of the nucleotide sequence of SEQ ID NO: 3. Specifically, any nucleotide sequence exhibiting a homology of 60% or more, 70% or more, preferably 80% or more, more preferably 90% or more, even more preferably 95% or more, and most preferably 99% or more with the sequence of SEQ ID NO: 3 may be included without limitation, as long as it is substantially capable of expressing a protein having 3HB-CoA dehydratase activity.

As used herein, the term “propionyl CoA-transferase” refers to an enzyme that converts crotonate into crotonyl-CoA.

The gene encoding propionyl CoA-transferase may be pct derived from Ralstonia eutropha, and may specifically consist of the nucleotide sequence of SEQ ID NO: 5. Specifically, any nucleotide sequence exhibiting a homology of 60% or more, 70% or more, preferably 80% or more, more preferably 90% or more, even more preferably 95% or more, and most preferably 99% or more with the sequence of SEQ ID NO: 5 may be included without limitation, as long as it is substantially capable of expressing a protein having propionyl CoA-transferase activity.

As used herein, the term “polynucleotide”, which is a polymer of nucleotides composed of nucleotide monomers connected in a lengthy chain by a covalently bond, is a DNA or RNA strand having at least a certain length. More specifically, it may refer to a polynucleotide fragment encoding the variant.

The polynucleotide of the present disclosure may undergo various modifications in the coding region within the scope that does not change the amino acid sequence of the variant of the present disclosure, due to codon degeneracy or in consideration of the codons preferred in an organism in which the variant of the present disclosure is to be expressed. Specifically, any polynucleotide sequences capable of encoding the enzymes involved in the poly(3-hydroxybutyrate-co-4-hydroxybutyrate) via crotonyl-CoA biosynthetic pathway via crotonyl-CoA are included in the scope of the present disclosure.

For example, in the present disclosure, although it is described as ‘a gene having a nucleotide sequence of a particular SEQ ID NO’, ‘a polynucleotide having a nucleotide sequence of a particular SEQ ID NO’, or ‘a polynucleotide including a nucleotide sequence of a particular SEQ ID NO’, it is apparent that any polynucleotide which has deletion, modification, substitution, conservative substitution or addition in part of the nucleotide sequence may also be used in the present disclosure, as long as the polynucleotide has the same or corresponding activity to the polynucleotide consisting of the nucleotide sequence of the corresponding SEQ ID NO.

For example, it is apparent that any polynucleotide in which a meaningless sequence is added to the inside or at the end of the nucleotide sequence of the corresponding SEQ ID NO, or a part of the sequence inside or at the end of the nucleotide sequence of the corresponding SEQ ID NO is deleted may fall within the scope of the present disclosure as long as the polynucleotide has the same or corresponding activity to the polynucleotide above.

As used herein, the term ‘homology’ or ‘identity’ refers to a degree of relevance between two given amino acid sequences or nucleotide sequences, and may be expressed as a percentage.

The terms homology and identity may often be used interchangeably with each other.

The sequence homology or identity of conserved polynucleotides may be determined by standard alignment algorithms and can be used with a default gap penalty established by the program being used. Substantially, homologous or identical sequences are generally expected to hybridize to all or at least about 50%, 60%, 70%, 80%, or 90% of the entire length of the sequences under moderate or high stringent conditions. Polynucleotides that contain degenerate codons instead of codons in hybridizing polynucleotides are also considered.

Whether any two polynucleotide sequences have a homology, similarity, or identity may be, for example, determined by a known computer algorithm such as the “FASTA” program (Pearson et al., (1988) [Proc. Natl. Acad. Sci. USA 85]: 2444) using default parameters. Alternatively, it may be determined by the Needleman-Wunsch algorithm (Needleman and Wunsch, 1970, J. Mol. Biol. 48: 443-453), which is performed using the Needleman program of the EMBOSS package (EMBOSS: The European Molecular Biology Open Software Suite, Rice et al., 2000, Trends Genet. 16: 276-277) (preferably, version 5.0.0 or versions thereafter) (GCG program package (Devereux, J., et al., Nucleic Acids Research 12: 387 (1984)), BLASTP, BLASTN, FASTA (Atschul, [S.] [F.,] [ET AL., J MOLEC BIOL 215]: 403 (1990); Guide to Huge Computers, Martin J. Bishop, [ED.,] Academic Press, San Diego, 1994, and [CARILLO ETA/.](1988) SIAM J Applied Math 48: 1073). For example, the homology, similarity, or identity may be determined using BLAST or ClustalW of the National Center for Biotechnology Information (NCBI).

The homology, similarity, or identity of polynucleotides may be determined by comparing sequence information using, for example, the GAP computer program, such as Needleman et al. (1970), J Mol Biol. 48: 443 as disclosed in Smith and Waterman, Adv. Appl. Math (1981) 2:482. In summary, the GAP program defines the homology, similarity, or identity as the value obtained by dividing the number of similarly aligned symbols (i.e., nucleotides or amino acids) by the total number of the symbols in the shorter of the two sequences. Default parameters for the GAP program may include (1) a unary comparison matrix (containing a value of 1 for identities and 0 for non-identities) and the weighted comparison matrix of Gribskov et al. (1986), Nucl. Acids Res. 14:6745, as disclosed in Schwartz and Dayhoff, eds., Atlas of Protein Sequence and Structure, National Biomedical Research Foundation, pp. 353-358 (1979) (or EDNAFULL substitution matrix (EMBOSS version of NCBI NUC4.4)); (2) a penalty of 3.0 for each gap and an additional 0.10 penalty for each symbol in each gap (or a gap opening penalty of 10 and a gap extension penalty of 0.5); and (3) no penalty for end gaps.

Accordingly, as used herein, the term “homology” or “identity” refers to the relevance between sequences.

Additionally, a probe that may be prepared from a known gene sequence, for example, any polynucleotide sequence which can hybridize with a sequence complementary to all or part of the polynucleotide sequence described above under stringent conditions may be included without limitation. The “stringent conditions” refers to conditions under which specific hybridization between polynucleotides is allowed. Such conditions are specifically described in the literature (J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York, 9.50-9.51, 11.7-11.8). For example, the stringent conditions may include conditions under which genes having a high homology or identity of 40% or more, specifically 60% or more, 70% or more, 80% or more, 85% or more, 90% or more, more specifically 95% or more, even more specifically 97% or more, or most specifically 99% or more are hybridized with each other and genes having a homology or identity lower than the above homologies or identities are not hybridized with each other, or washing conditions of Southern hybridization, that is, washing once, specifically, twice or three times at a salt concentration and a temperature corresponding to 60° C., 1×SSC, 0.1% SDS, specifically, 60° C., 0.1×SSC, 0.1% SDS, and more specifically 68° C., 0.1×SSC, 0.1% SDS.

Hybridization requires that two nucleic acids contain complementary sequences, although mismatches between bases are possible depending on the stringency of the hybridization. The term “complementary” is used to describe the relationship between nucleotide bases that can hybridize with each other. For example, with respect to DNA, adenine is complementary to thymine, and cytosine is complementary to guanine. Therefore, the polynucleotide of the present disclosure may include isolated nucleotide fragments complementary to the entire sequence as well as nucleic acid sequences substantially similar thereto.

Specifically, polynucleotides having a homology or identity with the polynucleotide may be detected using the hybridization conditions including a hybridization step at a Tₘ value of 55° C. under the above-described conditions. Further, the Tₘ value may be 60° C., 63° C., or 65° C., but is not limited thereto, and may be appropriately adjusted by those skilled in the art depending on the purpose thereof.

The appropriate stringency for hybridizing the polynucleotides depends on the length of the polynucleotides and the degree of complementation, and these variables are well known in the art (e.g., Sambrook et al.).

In one embodiment, the activity of enoyl-CoA hydratase and/or the activity of 4-hydroxybutyryl-CoA dehydratase may be regulated, but is not limited thereto.

In any one of the above-described embodiments, the activity of enoyl-CoA hydratase activity may be weakened, and/or the activity of 4-hydroxybutyryl-CoA dehydratase activity may be enhanced.

The production of 4-hydroxybutyrate through the TCA cycle is closely related to strain growth, which makes it difficult to regulate its content. Therefore, in order to regulate the 4-hydroxybutyrate content, the present inventors increased 4HB monomer production by relatively regulating the metabolic flux of the 4HB pathway and decreased 3HB monomer production by regulating the metabolic flux of the 3HB pathway using a novel pathway capable of producing both 3-hydroxybutyrate and 4-hydroxybutyrate from crotonyl-CoA, a common precursor, thereby producing poly(3-hydroxybutyrate-co-4-hydroxybutyrate) with various 4-hydroxybutyrate contents.

Specifically, it was confirmed that poly(3-hydroxybutyrate-co-4-hydroxybutyrate) with various 4-hydroxybutyrate contents could be produced by regulating the metabolic fluxes of the 3HB and 4HB pathways by weakening the activity of enoyl-CoA hydratase of the 3HB pathway and enhancing the activity of 4-hydroxybutyryl-CoA dehydratase of the 4HB pathway.

More specifically,

It was confirmed that poly(3-hydroxybutyrate-co-4-hydroxybutyrate) with various 4-hydroxybutyrate contents by regulating the expression intensity of the genes involved in each of the 3-hydroxybutyrate and 4-hydroxybutyrate biosynthetic pathways through crotonyl-CoA, a common precursor of 3-hydroxybutyrate and 4-hydroxybutyrate, specifically the gene encoding enoyl-CoA hydratase and/or the gene encoding 4-hydroxybutyryl-CoA dehydratase.

As used herein, the term “regulation” is a comprehensive concept that includes both “enhancement” or “weakening” of polypeptide activity to produce various contents of monomeric monomers in the target poly(3-hydroxybutyrate-co-4-hydroxybutyrate).

As used herein, the term “weakening” of the activity of a polypeptide is a comprehensive concept including both reduced or no activity compared to its endogenous activity. The weakening may be used interchangeably with terms such as inactivation, deficiency, down-regulation, decrease, reduce, attenuation, etc.

The weakening may also include a case where the polypeptide activity itself is decreased or removed compared to the activity of the polypeptide originally possessed by a microorganism due to a mutation of the polynucleotide encoding the polypeptide; a case where the overall level of intracellular polypeptide activity and/or concentration (expression level) is decreased compared to a natural strain due to the inhibition of expression of the gene of the polynucleotide encoding the polypeptide, or the inhibition of translation into the polypeptide, etc.; a case where the polynucleotide is not expressed at all; and/or a case where no polypeptide activity is observed even when the polynucleotide is expressed. As used herein, the term “endogenous activity” refers to the activity of a particular polypeptide originally possessed by a parent strain before transformation, a wild-type or a non-modified microorganism, when a trait is altered through genetic modification caused by natural or artificial factors, and may be used interchangeably with “activity before modification”. The expression that the polypeptide activity is “inactivated, deficient, decreased, down-regulated, reduced or attenuated” compared to its endogenous activity means that the polypeptide activity is decreased compared to the activity of a particular polypeptide originally possessed by a parent strain before transformation or a non-modified microorganism.

The weakening of the polypeptide activity can be performed by any method known in the art, but the method is not limited thereto, and can be achieved by applying various methods well known in the art (e.g., Nakashima N et al., Bacterial cellular engineering by genome editing and gene silencing. Int J Mol Sci. 2014; 15(2):2773-2793, Sambrook et al. Molecular Cloning 2012, etc.).

Specifically, the weakening of the polypeptide activity of the present disclosure may be achieved by:
1) deleting a part or all of the gene encoding the polypeptide; or decreasing the intracellular copy number of a polynucleotide encoding the polypeptide; 2) modifying the expression regulatory region (expression regulatory sequence) such that the expression of the gene encoding the polypeptide is decreased;
3) modifying the amino acid sequence constituting the polypeptide such that the polypeptide activity is removed or weakened (e.g., deletion/substitution/addition of one or more amino acids on the amino acid sequence);
4) modifying the gene sequence encoding the polypeptide such that the polypeptide activity is removed or weakened (e.g., deletion/substitution/addition of one or more of nucleotides on the nucleotide sequence of the polypeptide gene to encode a polypeptide that has been modified to remove or weaken the activity of the polypeptide);
5) modifying the nucleotide sequence encoding the initiation codon or 5′-UTR of the gene transcript encoding the polypeptide;
6) introducing an antisense oligonucleotide (e.g., antisense RNA), which binds complementary to the gene transcript encoding the polypeptide;
7) adding a sequence complementary to the Shine-Dalgarno (SD) sequence on the front end of the SD sequence of the gene encoding the polypeptide to form a secondary structure, thereby inhibiting the ribosomal attachment;
8) a reverse transcription engineering (RTE), which adds a promoter, which is to be reversely transcribed, on the 3′ terminus of the open reading frame (ORF) of the gene sequence encoding the polypeptide; or
9) a combination of two or more selected from the methods 1) to 8) above, but is not particularly limited thereto.

For example,
The 1) method of deleting a part or all of the gene encoding the polypeptide may be achieved by deleting all of the polynucleotide encoding the endogenous target polypeptide within the chromosome, or by replacing the polynucleotide with a polynucleotide having a partially deleted nucleotide, or with a marker gene.
The 2) method of modifying the expression regulatory region (expression regulatory sequence) may be achieved by inducing a modification on the expression regulatory region (expression regulatory sequence) through deletion, insertion, non-conservative substitution or conservative substitution, or a combination thereof; or by replacing the sequence with a sequence having a weaker activity. The expression regulatory region may include a promoter, an operator sequence, a sequence encoding a ribosome-binding site, and a sequence for regulating the termination of transcription and translation, but is not limited thereto. For example, it may be achieved by replacing the original promoter with a weaker promoter, but is not limited thereto.
The 5) method of modifying the nucleotide sequence encoding the initiation codon or 5′-UTR of the gene transcript encoding the polypeptide may be achieved, for example, by substituting the nucleotide sequence with a nucleotide sequence encoding another initiation codon having a lower polypeptide expression rate than the endogenous initiation codon, but is not limited thereto.
The 3) and 4) methods of modifying the amino acid sequence or the polynucleotide sequence may be achieved by inducing a modification on the sequence through deletion, insertion, non-conservative or conservative substitution of the amino acid sequence of the polypeptide or the polynucleotide sequence encoding the polypeptide, or a combination thereof to weaken the activity of the polypeptide, or by replacing the sequence with an amino acid sequence or a polynucleotide sequence modified to have a weaker activity, or an amino acid sequence or a polynucleotide sequence modified to have no activity, but are not limited thereto. For example, the expression of the gene may be inhibited or weakened by introducing a mutation into the polynucleotide sequence to form a termination codon, but is not limited thereto.
The 6) method of introducing an antisense oligonucleotide (e.g., antisense RNA), which binds complementary to the gene transcript encoding the polypeptide can be found in the literature [Weintraub, H. et al., Antisense-RNA as a molecular tool for genetic analysis, Reviews—Trends in Genetics, Vol. 1(1) 1986].
The 7) method of adding a sequence complementary to the Shine-Dalgarno (SD) sequence on the front end of the SD sequence of the gene encoding the polypeptide to form a secondary structure, thereby inhibiting the ribosome attachment may be achieved by inhibiting mRNA translation or reducing the speed thereof.
The 8) reverse transcription engineering (RTE), which adds a promoter, which is to be reversely transcribed, on the 3′ terminus of the open reading frame (ORF) of the gene sequence encoding the polypeptide may be achieved by forming an antisense nucleotide complementary to the gene transcript encoding the polypeptide to weaken the activity.

In one embodiment, the activity of enoyl-CoA hydratase may be weakened by a method selected from the group consisting of reduction in intracellular copy number of a polynucleotide encoding a polypeptide, replacement of a promoter, genomic insertion of a gene encoding a polypeptide, and a combination thereof, but is not limited thereto.

As used herein, the term “enhancement” of a polypeptide activity means that the activity of a polypeptide is increased compared to its endogenous activity. The enhancement may be used interchangeably with terms such as activation, up-regulation, overexpression, increase, etc. In particular, the activation, enhancement, up-regulation, overexpression and increase may include both cases in which an activity not originally possessed is exhibited, or the activity is enhanced compared to the endogenous activity or the activity before modification. The “endogenous activity” refers to the activity of a particular polypeptide originally possessed by a parent strain before transformation or a non-modified microorganism, when a trait is altered through genetic modification caused by natural or artificial factors, and may be used interchangeably with “activity before modification”. The “enhancement”, “up-regulation”, “overexpression” or “increase” in the activity of a polypeptide compared to its endogenous activity means that the activity and/or concentration (expression level) of the polypeptide is enhanced compared to that of a particular polypeptide originally possessed by a parent strain before transformation or a non-modified microorganism.

The enhancement may be achieved by introducing a foreign polypeptide, or by enhancing the activity and/or concentration (expression level) of the endogenous polypeptide. The enhancement of the activity of the polypeptide can be confirmed by the increase in the level of activity of the polypeptide, expression level, or the amount of product excreted from the polypeptide.

The enhancement of the activity of the polypeptide can be applied by various methods well known in the art, and is not limited as long as it can enhance the activity of the target polypeptide compared to that of the microorganism before modification. Specifically, genetic engineering and/or protein engineering well known to those skilled in the art, which is a common method of molecular biology, may be used, but the method is not limited thereto (e.g., Sitnicka et al. Functional Analysis of Genes. Advances in Cell Biology. 2010, Vol. 2. 1-16, Sambrook et al. Molecular Cloning 2012, etc.).

Specifically, the enhancement of the polypeptide of the present disclosure may be achieved by:
1) increasing the intracellular copy number of a polynucleotide encoding the polypeptide;
2) replacing the expression regulatory region of a gene on the chromosome with a sequence having a stronger activity;
3) modifying a nucleotide sequence encoding the initiation codon or 5′-UTR of the gene transcript encoding the polypeptide;
4) modifying the amino acid sequence of the polypeptide such that the activity of the polypeptide is enhanced;
5) modifying the polynucleotide sequence encoding the polypeptide such that the activity of the polypeptide is enhanced (e.g., modifying the polynucleotide sequence of the polypeptide gene to encode a polypeptide that has been modified to enhance the activity of the polypeptide);
6) introducing a foreign polypeptide exhibiting the polypeptide activity or a foreign polynucleotide encoding the same;
7) codon-optimization of the polynucleotide encoding the polypeptide;
8) analyzing the tertiary structure of the polypeptide and thereby selecting and modifying the exposed site, or chemically modifying the same; or
9) a combination of two or more selected from above 1 to 8), but is not particularly limited thereto.

More specifically,
The 1) method of increasing the intracellular copy number of a polynucleotide encoding the polypeptide may be achieved by introducing a vector, which is operably linked to the polynucleotide encoding the polypeptide and is able to replicate and function regardless of a host cell, into the host cell. Alternatively, the method may be achieved by introducing one copy or two copies of polynucleotides encoding the polypeptide into the chromosome of a host cell. The introduction into the chromosome may be performed by introducing a vector, which is able to insert the polynucleotide into the chromosome of a host cell, into the host cell, but is not limited thereto.
The 2) method of replacing the expression regulatory region (or expression regulatory sequence) of a gene encoding the polypeptide on the chromosome with a sequence having a strong activity may be achieved, for example, by inducing a modification on the sequence through deletion, insertion, non-conservative or conservative substitution, or a combination thereof to further enhance the activity of the expression regulatory region, or by replacing the sequence with a sequence having a stronger activity. The expression regulatory region may include, but is not particularly limited to, a promoter, an operator sequence, a sequence encoding a ribosome-binding site, and a sequence regulating the termination of transcription and translation, etc. In one example, the method may include replacing the original promoter with a strong promoter, but is not limited thereto.

Examples of the known strong promoter may include CJ1 to CJ7 promoters (U.S. Pat. No. 7,662,943 B2), lac promoter, trp promoter, trc promoter, tac promoter, lambda phage PR promoter, PL promoter, tet promoter, gapA promoter, SPL7 promoter, SPL13 (sm3) promoter (U.S. Ser. No. 10/584,338 B2), O2 promoter (U.S. Ser. No. 10/273,491 B2), tkt promoter, yccA promoter, etc., but the strong promoter is not limited thereto.
The 3) method of modifying a nucleotide sequence encoding the initiation codon or 5′-UTR of the gene transcript encoding the polypeptide may be achieved, for example, by substituting the nucleotide sequence with a nucleotide sequence encoding another initiation codon having a higher expression rate of the polypeptide compared to the endogenous initiation codon, but is not limited thereto.
The 4) and 5) methods of modifying the amino acid sequence or the polynucleotide sequence may be achieved by inducing a modification on the sequence through deletion, insertion, non-conservative or conservative substitution of the amino acid sequence of the polypeptide or the polynucleotide sequence encoding the polypeptide, or a combination thereof to enhance the activity of the polypeptide, or by replacing the sequence with an amino acid sequence or polynucleotide sequence modified to have a stronger activity, or an amino acid sequence or polynucleotide sequence modified to enhance the activity, but are not limited thereto. The replacement may specifically be performed by inserting the polynucleotide into the chromosome by homologous recombination, but is not limited thereto. The vector used herein may further include a selection marker to confirm the insertion into the chromosome. The selection marker is as described above.
The 6) method of introducing a foreign polynucleotide exhibiting the activity of the polypeptide may be achieved by introducing into a host cell a foreign polynucleotide encoding a polypeptide that exhibits the same/similar activity to that of the polypeptide. The foreign polynucleotide may be used without limitation regardless of its origin or sequence as long as it exhibits the same/similar activity to that of the polypeptide. The introduction may be performed by those of ordinary skill in the art by appropriately selecting a transformation method known in the art, and the expression of the introduced polynucleotide in the host cell enables to produce the polypeptide, thereby increasing its activity.
The 7) method of codon-optimization of the polynucleotide encoding the polypeptide may be achieved by codon-optimization of an endogenous polynucleotide to increase the transcription or translation within a host cell, or by optimizing the codons thereof such that the optimized transcription and translation of the foreign polynucleotide can be achieved within the host cell.
The 8) method of analyzing the tertiary structure of the polypeptide and thereby selecting and modifying the exposed site, or chemically modifying the same may be achieved, for example, by comparing the sequence information of the polypeptide to be analyzed with a database, in which the sequence information of known proteins is stored, to determine template protein candidates according to the degree of sequence similarity, and thus confirming the structure based on the information, thereby selecting and transforming or modifying the exposed site to be modified or chemically modified.

Such enhancement of the polypeptide activity may mean that the activity or concentration of the corresponding polypeptide is increased relative to the activity or concentration of the polypeptide expressed in a wild-type or a microorganism before modification, or that the amount of product produced from the polypeptide is increased, but is not limited thereto.

In one embodiment, the activity of 4-hydroxybutyryl-CoA dehydratase may be enhanced by replacing the original promoter with a stronger promoter, but is not limited thereto.

In one embodiment, the poly(3-hydroxybutyrate-co-4-hydroxybutyrate) may have a 4-hydroxybutyrate content of 0.1% to 60%, specifically 0.5% to 20%, 2% to 15%, 2.1% to 13%, 2.2% to 12.5%, or 3% to 12.2%, but is not limited thereto.

In one example, the poly(3-hydroxybutyrate-co-4-hydroxybutyrate) may have a 4-hydroxybutyrate content of about 0.1% or more, about 0.5% or more, about 1% or more, about 2% or more, about 2.5% or more, about 3% or more, about 3.5% or more, about 4% or more, about 4.5% or more, about 5% or more, about 5.5% or more, about 6% or more, about 6.5% or more, about 7% or more, about 7.5% or more, about 8% or more, about 8.5% or more, about 9% or more, about 9.5% or more, about 10% or more, about 10.5% or more, about 11% or more, about 11.5% or more, about 12% or more, about 12.5% or more, about 13% or more, about 13.5% or more, about 14% or more, about 14.5% or more, about 15% or more (the upper limit is not particularly limited, for example, about 200% or less, about 150% or less, about 100% or less, about 90% or less, about 80% or less, about 70% or less, about 60% or less), but is not limited thereto. As used herein, the term “about” refers to a range which includes all of ±0.5, ±0.4, ±0.3, ±0.2, ±0.1, etc. and includes all of the values that are equivalent or similar to those following the values, but the range is not limited thereto.

The method may further include purifying the prepared poly(3-hydroxybutyrate-co-4-hydroxybutyrate). The purification method is not particularly limited, and any method commonly used in the technical field of the present disclosure may be used. Non-limiting examples include chromatography, fractional crystallization, and ion purification. One purification method may be performed, and two or more methods may be performed in combination. In one example, the method for preparing poly(3-hydroxybutyrate-co-4-hydroxybutyrate) of the present disclosure includes both a recovering step and a purification step, the recovering step and the purification step may be performed continuously or intermittently regardless of the order or simultaneously, or may be integrated into one step, but the method is not limited thereto.

Another aspect of the present disclosure provides a recombinant vector containing the genes involved in the biosynthetic pathway of the poly(3-hydroxybutyrate-co-4-hydroxybutyrate) via crotonyl-CoA of the present disclosure.

As used herein, the term “the genes involved in the biosynthetic pathway of the poly(3-hydroxybutyrate-co-4-hydroxybutyrate) via crotonyl-CoA of the present disclosure” refers to a plurality of genes each encoding enzymes involved in the biosynthetic pathway of the poly(3-hydroxybutyrate-co-4-hydroxybutyrate) via crotonyl-CoA of the present disclosure. In one example, it may include a gene encoding enoyl-CoA hydratase, a gene encoding 4-hydroxybutyryl-CoA dehydratase, and a gene encoding PHA synthase, and may further include: (i) a gene encoding acetyl-CoA acetyltransferase, a gene encoding 3-hydroxybutyryl-CoA dehydrogenase, and a gene encoding 3HB-CoA dehydratase; or (ii′) a gene encoding propionyl CoA-transferase.

In one embodiment, the genes involved in the biosynthetic pathway of the poly(3-hydroxybutyrate-co-4-hydroxybutyrate) via crotonyl-CoA of the present disclosure may be of foreign origin, but is not limited thereto.

The genes involved in the biosynthetic pathway of the poly(3-hydroxybutyrate-co-4-hydroxybutyrate) via crotonyl-CoA of the present disclosure may be those in which a part of the plurality of genes each encoding enzymes involved in the biosynthetic pathway of the poly(3-hydroxybutyrate-co-4-hydroxybutyrate) via crotonyl-CoA of the present disclosure is enhanced or weakened.

As used herein, the term “vector” is an artificial DNA molecule that possesses a genetic material to enable the expression of a target gene in an appropriate host cell, and specifically refers to a DNA construct which includes the nucleotide sequence of a gene encoding a target protein operably linked thereto.

As used herein, the term “operably linked” means that the polynucleotide having the promoter activity of the present disclosure and the gene sequence are functionally linked so that the transcription of the target gene can be initiated and mediated. The operable linkage may be prepared using a genetic recombinant technology well-known in the art, and site-specific DNA cleavage and linkage may be prepared using cleavage and linking enzymes, etc., known in the art, but is not limited thereto.

The vector used in the present disclosure may not be particularly limited as long as the vector is expressible in a host cell, and the host cell may be transformed using any vector known in the art. Examples of the conventionally-used vector may include natural or recombinant plasmids, cosmids, viruses, and bacteriophages.

For example, as a phage vector or cosmid vector, pWE15, M13, MBL3, MBL4, IXII, ASHII, APII, t10, t11, Charon4A, Charon21A, etc., may be used; and as a plasmid vector, those based on pDZ, pBR, pUC, pBluescriptII, pGEM, pTZ, pCL, pET, etc., may be used, but the vector is not limited thereto. Specifically, pDZ, pDC, pDCM2, pACYC177, pACYC184, pCL, pECCG117, pUC19, pBR322, pMW118, pCC1BAC vectors, etc., may be used, but the vector is not limited thereto. The insertion of the polynucleotide into chromosome may be carried out by a method well-known in the art, e.g., homologous recombination.

Since the vector of the present disclosure can be inserted into the chromosome via homologous recombination, a selection marker for confirming the insertion into the chromosome may further be included. The selection marker is used for the selection of a transformed cell, i.e., for confirming the insertion of the polynucleotide, and markers capable of providing selectable phenotypes such as drug resistance, nutrient requirement, resistance to cytotoxic agents, or expression of surface proteins may be used. Under the circumstances where selective agents are treated, only the cells capable of expressing the selection markers can survive or express other phenotypic traits, and thus the transformed cells can be selected.

As used herein, the term “transformation” refers to a process for introducing a vector including a polynucleotide encoding a target protein into a host cell, thereby enabling the expression of the protein encoded by the polynucleotide in the host cell. For the transformed polynucleotide, it does not matter whether it is inserted into the chromosome of a host cell and located therein or located outside the chromosome, and both cases can be included, as long as it can be expressed in the host cell. Additionally, the polynucleotide may include DNA and RNA encoding the target protein, and may be introduced in any form, as long as it can be introduced into a host cell and expressed therein. For example, the polynucleotide may be introduced in the form of an expression cassette, which is a gene construct including all of the essential elements required for self-expression, or in the form of a vector including the expression cassette.

The method of transformation may include any method which can introduce a gene encoding the target protein into a cell, and the transformation may be performed by selecting an appropriate standard technique as known in the art according to the host cell. For example, the method may include electroporation, calcium phosphate (CaPO₄) precipitation, calcium chloride (CaCl₂)) precipitation, microinjection, a polyethylene glycol (PEG) method, a DEAE-dextran method, a cationic liposome method, and a lithium acetate-DMSO method, etc., but is not limited thereto.

Still another aspect of the present disclosure provides a microorganism having poly(3-hydroxybutyrate-co-4-hydroxybutyrate) productivity, including a gene encoding enoyl-CoA hydratase, a gene encoding 4-hydroxybutyryl-CoA dehydratase, and a gene encoding PHA synthase.

As used herein, the term “microorganism (or strain)” includes all wild-type microorganisms, or naturally or artificially genetically modified microorganisms, and it may be a microorganism in which a particular mechanism is weakened or enhanced due to insertion of a foreign gene, or enhancement or inactivation of the activity of an endogenous gene, etc., and may be a microorganism including genetic modification to produce a desired polypeptide, protein or product.

As used herein, the term “microorganism having poly(3-hydroxybutyrate-co-4-hydroxybutyrate) productivity” is a prokaryotic or eukaryotic microbial strain capable of producing poly(3-hydroxybutyrate-co-4-hydroxybutyrate) in an organism, and may include all of the microorganism that have been given the ability of producing poly(3-hydroxybutyrate-co-4-hydroxybutyrate) to a parent strain that does not have the ability of producing poly(3-hydroxybutyrate-co-4-hydroxybutyrate), or microorganisms endogenously having the ability of producing poly(3-hydroxybutyrate-co-4-hydroxybutyrate), but is not limited thereto. The ability of producing poly(3-hydroxybutyrate-co-4-hydroxybutyrate) may be imparted or enhanced by species modification.

The microorganism producing poly(3-hydroxybutyrate-co-4-hydroxybutyrate) of the present disclosure may include the genes involved in the biosynthetic pathway of crotonyl-CoA-based poly(3-hydroxybutyrate-co-4-hydroxybutyrate) of the present disclosure and/or a recombinant vector containing the genes involved in the biosynthetic pathway of the poly(3-hydroxybutyrate-co-4-hydroxybutyrate) via crotonyl-CoA of the present disclosure, but is not limited thereto. Additionally, the genes involved in the biosynthetic pathway of the poly(3-hydroxybutyrate-co-4-hydroxybutyrate) via crotonyl-CoA and the recombinant vector may be introduced into the microorganism by transformation, but is not limited thereto.

Specifically, the microorganism is characterized in producing desired poly(3-hydroxybutyrate-co-4-hydroxybutyrate) having various 4-hydroxybutyrate contents by introducing the genes involved in the biosynthetic pathway of the poly(3-hydroxybutyrate-co-4-hydroxybutyrate) via crotonyl-CoA and/or the recombinant vector, and the microorganism may genetically modified microorganism or a recombinant microorganism, but is not limited thereto.

As used herein, the term “introduction” means that a microorganism exhibits an activity of a particular protein, or the microorganism exhibits enhanced activity compared to its endogenous activity or the activity of the protein before modification, as a gene which was not originally possessed by the microorganism is expressed in the microorganism. For example, it may mean that a polynucleotide encoding a particular protein is introduced into the chromosome of a microorganism; or a vector containing a polynucleotide encoding a particular protein is introduced into a microorganism and thereby allows the activity of the particular protein to be exhibited.

As used herein, the term “non-modified microorganism” does not exclude a strain containing a mutation that may occur naturally in a microorganism, and may be a wild-type strain itself, or a strain before the trait is altered due to genetic modification caused by natural or artificial factors. For example, the non-modified microorganism may refer to a strain into which the genes involved in the biosynthetic pathway of the poly(3-hydroxybutyrate-co-4-hydroxybutyrate) via crotonyl-CoA of the present disclosure are not introduced, or a strain before the introduction of the genes involved in the biosynthetic pathway of the poly(3-hydroxybutyrate-co-4-hydroxybutyrate) via crotonyl-CoA. The “non-modified microorganism” may be used interchangeably with “strain before modification”, “microorganism before modification”, “non-mutant strain”, “non-modified strain”, “non-mutant microorganism” or “reference microorganism”.

The microorganism having poly(3-hydroxybutyrate-co-4-hydroxybutyrate) productivity may be any prokaryotic or eukaryotic microorganism, and specifically, prokaryotic microorganism. The prokaryotic microorganism may, for example, include microbial strains belonging to the genus Escherichia, the genus Erwinia, the genus Serratia, the genus Providencia, the genus Corynebacterium, the genus Pseudomonas, the genus Leptospira, the genus Salmonella, the genus Brevibacteria, the genus Hypomononas, the genus Chromobacterium, and the genus Norcardia, or fungi or yeasts. Specifically, the microorganism may be a microbial strain belonging to the genus Escherichia, the genus Corynebacterium, the genus Leptospira, and yeasts. More specifically, it may be a microbial strain belonging to the genus Escherichia, and more specifically Escherichia coli, but is not limited thereto.

In one embodiment, the gene encoding the enoyl-CoA hydratase may be derived from Aeromonas caviae; the gene encoding the 4-hydroxybutyryl-CoA dehydratase may be derived from Nitrosopumilus maritimus, Candidatus Nitrosopelagicus brevis, Candidatus Nitrosarchaeum limnium, or Thaumarchaeota archaeon; and the gene encoding the PHA synthase may be derived from Ralstonia eutropha, but the genes are not limited thereto.

In one embodiment, the microorganism may further include:
(i′) a gene encoding acetyl-CoA acetyltransferase, a gene encoding 3-hydroxybutyryl-CoA dehydrogenase, and a gene encoding 3HB-CoA dehydratase; or
(ii′) a gene encoding propionyl CoA-transferase, but is not limited thereto.

In one embodiment, the gene encoding the acetyl-CoA acetyltransferase may be derived from Ralstonia eutropha; the gene encoding the 3-hydroxybutyryl-CoA dehydrogenase may be derived from Clostridium acetobutylicum; the gene encoding the 3HB-CoA dehydratase may be derived from Clostridium acetobutylicum; and the gene encoding the propionyl CoA-transferase may be derived from Ralstonia eutropha, but the genes are not limited thereto.

The microorganism producing poly(3-hydroxybutyrate-co-4-hydroxybutyrate) of the present disclosure may be those in which a part of the genes in the biosynthetic pathway of the poly(3-hydroxybutyrate-co-4-hydroxybutyrate) via crotonyl-CoA is enhanced or weakened.

In one embodiment, the microorganism having poly(3-hydroxybutyrate-co-4-hydroxybutyrate) productivity may be those in which the activity of enoyl-CoA hydratase and/or the activity of 4-hydroxybutyryl-CoA dehydratase is regulated, but is not limited thereto.

In any one of the above-described embodiments, the microorganism having poly(3-hydroxybutyrate-co-4-hydroxybutyrate) productivity of the present disclosure may be those in which the activity of enoyl-CoA hydratase is weakened and/or the activity of 4-hydroxybutyryl-CoA dehydratase is enhanced, but is not limited thereto.

In one embodiment, the poly(3-hydroxybutyrate-co-4-hydroxybutyrate) may have a 4-hydroxybutyrate content of 0.1% to 60%, specifically 0.5% to 20%, 2% to 15%, 2.1% to 13%, 2.2% to 12.5%, or 3% to 12.2%, but the content is not limited thereto.

The microorganism of the present disclosure may produce poly(3-hydroxybutyrate-co-4-hydroxybutyrate) by culturing the microorganism, but is not limited thereto.

As used herein, the term “cultivation” means that the microorganism of the present disclosure is grown under appropriately controlled environmental conditions. The cultivation process of the present disclosure may be performed in a suitable culture medium and culture conditions known in the art. Such a cultivation process may be easily adjusted for use by those skilled in the art according to the strain to be selected. Specifically, the cultivation may be a batch culture, a continuous culture, and a fed-batch culture, but is not limited thereto.

As used herein, the term “medium” refers to a mixture of materials which contains nutrient materials required for the cultivation of the microorganism of the present disclosure as a main ingredient, and it supplies nutrient materials and growth factors, along with water that is essential for survival and growth. Specifically, the medium and other culture conditions used for culturing the microorganism of the present disclosure may be any medium used for conventional cultivation of microorganisms without any particular limitation. However, the microorganism of the present disclosure may be cultured under aerobic conditions in a conventional medium containing an appropriate carbon source, nitrogen source, phosphorus source, inorganic compound, amino acid, and/or vitamin, while adjusting temperature, pH, etc.

In the present disclosure, the carbon source may include carbohydrates, such as glucose, saccharose, lactose, fructose, sucrose, maltose, etc.; sugar alcohols, such as mannitol, sorbitol, etc.; organic acids, such as pyruvic acid, lactic acid, citric acid, etc.; amino acids, such as glutamic acid, methionine, lysine, etc. Additionally, the carbon source may include natural organic nutrients such as starch hydrolysate, molasses, blackstrap molasses, rice bran, cassava, sugar cane molasses, and corn steep liquor, etc. Specifically, carbohydrates such as glucose and sterilized pretreated molasses (i.e., molasses converted to reducing sugar) may be used, and in addition, various other carbon sources in an appropriate amount may be used without limitation. These carbon sources may be used alone or in a combination of two or more kinds, but are not limited thereto.

The nitrogen source may include inorganic nitrogen sources, such as ammonia, ammonium sulfate, ammonium chloride, ammonium acetate, ammonium phosphate, ammonium carbonate, ammonium nitrate, etc.; amino acids, such as glutamic acid, methionine, glutamine, etc.; and organic nitrogen sources, such as peptone, NZ-amine, meat extract, yeast extract, malt extract, corn steep liquor, casein hydrolysate, fish or decomposition product thereof, defatted soybean cake or decomposition product thereof, etc. These nitrogen sources may be used alone or in a combination of two or more kinds, but are not limited thereto.

The phosphorus source may include monopotassium phosphate, dipotassium phosphate, or corresponding sodium-containing salts, etc. Examples of the inorganic compound may include sodium chloride, calcium chloride, iron chloride, magnesium sulfate, iron sulfate, manganese sulfate, calcium carbonate, etc. Additionally, amino acids, vitamins, and/or appropriate precursors may be included. These constituting ingredients or precursors may be added to a medium in a batch or continuous manner, but these phosphorus sources are not limited thereto.

Additionally, the pH of the medium may be adjusted by adding a compound such as ammonium hydroxide, potassium hydroxide, ammonia, phosphoric acid, sulfuric acid, etc. during the cultivation of the microorganism in an appropriate manner. Additionally, bubble formation may be prevented during the cultivation using an antifoaming agent such as fatty acid polyglycol ester. Further, oxygen gas or a gas containing oxygen may be injected to the medium order to maintain aerobic conditions of the medium; or nitrogen gas, hydrogen gas, or carbon dioxide may be injected to maintain anaerobic or microaerobic conditions, without the injection of gas, but the gas is not limited thereto.

The temperature in the cultivation of the present disclosure may be in the range from 20° C. to 40° C., and more specifically from 28° C. to 37° C., but is not limited thereto. The cultivation may be continued until a desired amount of the target material is obtained, and may be specifically carried out for 10 to 100 hours.

The poly(3-hydroxybutyrate-co-4-hydroxybutyrate) produced by the cultivation of the present disclosure may be released into the medium or remain in the cells.

The method of preparing poly(3-hydroxybutyrate-co-4-hydroxybutyrate) using the microorganism of the present disclosure may further include a step of preparing the microorganism of the present disclosure, a step of preparing a medium for culturing the microorganism, or a combination thereof (regardless of the order, in any order), for example, prior to the culturing step.

The method of preparing poly(3-hydroxybutyrate-co-4-hydroxybutyrate) using the microorganism of the present disclosure may further include a step of recovering poly(3-hydroxybutyrate-co-4-hydroxybutyrate) from the culture medium (medium on which the culture was grown) or the microorganism of the present disclosure. The recovering step may be further included after the culturing step.

In the recovering step, the desired poly(3-hydroxybutyrate-co-4-hydroxybutyrate) may be collected using the method of culturing the microorganism of the present disclosure, for example, using a suitable method known in the art according to a batch culture, continuous culture, or fed-batch culture method. For example, methods such as centrifugation, filtration, treatment with a protein crystallizing precipitant (salting-out method), extraction, ultrasonic disruption, ultrafiltration, dialysis, various kinds of chromatographies such as molecular sieve chromatography (gel filtration), adsorption chromatography, ion exchange chromatography, affinity chromatography, etc., HPLC or a combination thereof may be used, and the desired poly(3-hydroxybutyrate-co-4-hydroxybutyrate) can be recovered from the medium or the microorganisms using suitable methods known in the art.

Yet another aspect of the present disclosure provides the use of a microorganism having poly(3-hydroxybutyrate-co-4-hydroxybutyrate) productivity, which includes the gene encoding enoyl-CoA hydratase, the gene encoding 4-hydroxybutyryl-CoA dehydratase, and the gene encoding PHA synthase, in the production of poly(3-hydroxybutyrate-co-4-hydroxybutyrate).

Even another aspect of the present disclosure provides a composition for producing poly(3-hydroxybutyrate-co-4-hydroxybutyrate), including: enoyl-CoA hydratase, 4-hydroxybutyryl-CoA dehydratase, and PHA synthase; a microorganism expressing the same; or a culture of the microorganism.

In one embodiment, the composition may further include:
(i″) acetyl-CoA acetyltransferase, 3-hydroxybutyryl-CoA dehydrogenase, and 3HB-CoA dehydratase; a microorganism expressing the same; or a culture of the microorganism; or
(ii″) propionyl CoA-transferase; a microorganism expressing the same; or a culture of the microorganism, but is not limited thereto.

The composition of the present invention may individually include two or more enzymes involved in the biosynthetic pathway of the poly(3-hydroxybutyrate-co-4-hydroxybutyrate) via crotonyl-CoA of the present disclosure or transformants thereof, or transformants transformed with nucleotides encoding two or more enzymes above.

In one embodiment, the poly(3-hydroxybutyrate-co-4-hydroxybutyrate) may have a 4-hydroxybutyrate content of 0.1% to 60%, specifically, 0.5% to 20%, 2% to 15%, 2.1% to 13%, 2.2% to 12.5%, or 3% to 12.2%, but is not limited thereto.

The composition of the present disclosure may further include any suitable excipient commonly used in compositions for producing poly(3-hydroxybutyrate-co-4-hydroxybutyrate), and such excipients include, for example, preservatives, wetting agents, dispersing agents, suspending agents, buffers, stabilizers, or isotonic agents, etc., but are not limited thereto.

Further another aspect of the present disclosure provides a method for regulating the 4-hydroxybutyrate content of poly(3-hydroxybutyrate-co-4-hydroxybutyrate), including:
(a) converting crotonyl-CoA into 3-hydroxybutyrate using enoyl-CoA hydratase and/or converting crotonyl-CoA into 4-hydroxybutyrate using 4-hydroxybutyryl-CoA dehydratase;
(b) preparing poly(3-hydroxybutyrate-co-4-hydroxybutyrate) by polymerization of 3-hydroxybutyrate and 4-hydroxybutyrate using PHA synthase; and
regulating the activity of enoyl-CoA hydratase and/or the activity of 4-hydroxybutyryl-CoA dehydratase, prior to step (a) above.

In one embodiment, the method for regulating the 4-hydroxybutyrate content of poly(3-hydroxybutyrate-co-4-hydroxybutyrate) of the present disclosure may further include:
(a) converting crotonyl-CoA into 3-hydroxybutyrate using enoyl-CoA hydratase and/or converting crotonyl-CoA into 4-hydroxybutyrate using 4-hydroxybutyryl-CoA dehydratase;
(b) preparing poly(3-hydroxybutyrate-co-4-hydroxybutyrate) by polymerization of 3-hydroxybutyrate and 4-hydroxybutyrate using PHA synthase; and
weakening the activity of enoyl-CoA hydratase and/or enhancing the activity of 4-hydroxybutyryl-CoA dehydratase, prior to step (a) above, but is not limited thereto.

In one embodiment, the poly(3-hydroxybutyrate-co-4-hydroxybutyrate) may have a 4-hydroxybutyrate content of 0.1% to 60%, specifically 0.5% to 20%, 2% to 15%, 2.1% to 13%, 2.2% to 12.5%, or 3% to 12.2%, but is not limited thereto.

In one embodiment, the step of regulating the activity of enoyl-CoA hydratase, in one example, the step of weakening the activity of enoyl-CoA hydratase, may be performed by a method selected from the group consisting of reduction in intracellular copy number of a polynucleotide encoding a polypeptide, replacement of a promoter, genomic insertion of a gene encoding a polypeptide, and a combination thereof, but is not limited thereto.

### Advantageous Effects

It is possible to effectively produce poly(3-hydroxybutyrate-co-4-hydroxybutyrate) having various 4-hydroxybutyrate contents when the method or microorganism of the present disclosure is used.

### BRIEF DESCRIPTION OF DRAWINGS

FIG. 1 is a diagram showing a novel biosynthetic pathway of poly(3-hydroxybutyrate-co-4-hydroxybutyrate) via crotonyl-CoA.

FIG. 2 shows the results of evaluating the ability to produce poly(3-hydroxybutyrate-co-4-hydroxybutyrate) of strains containing genes involved in the biosynthetic pathway of the poly(3-hydroxybutyrate-co-4-hydroxybutyrate) via crotonyl-CoA.

### MODE FOR CARRYING OUT THE INVENTION

Hereinafter, the present disclosure will be described in detail by way of Examples. However, these Examples are merely preferred Examples given for illustrative purposes, and thus, the scope of the present disclosure is not intended to be limited to or by these Examples. Meanwhile, technical features which are not described herein can be sufficiently understood and easily carried out by those skilled in the art in the technical field of the present disclosure or in a similar technical field.

### Plasmids

Plasmids used in Examples 1 to 5 are shown in Table 1 below.

TABLE 1

| Plasmid | Description |
|---|---|
| pSKH130 | R6K origin, sacB gene; Km^{R} |
| pSKYP1 | pSKH130 derivative (US 20200048642 A1); ΔmaeB |
| pSKYP2 | pSKH130 derivative (US 20200048642 A1); sacB gene, Px promoter |
| | (U.S. Pat. No. 10,323,261 B2), phaJ gene |
| pPYS10 | pCL1920 derivative (Lerner C G, Inouye M. Low copy number |
| | plasmids for regulated low-level expression of cloned genes in |
| | Escherichia coli with blue/white insert screening capability. Nucleic |
| | Acids Res. (1990) 18(15): 4631.); Px promoter (U.S. Pat. No. |
| | 10,323,261 B2), phaC gene |
| pPYS11 | pCL1920 derivative (Lerner C G, Inouye M. Low copy number |
| | plasmids for regulated low-level expression of cloned genes in |
| | Escherichia coli with blue/white insert screening capability. Nucleic |
| | Acids Res. (1990) 18(15): 4631.); PuspA promoter (Prytz et al. |
| | (2003); Dyk et al. (1995); Nyström and Neidhardt (1992)), phaA |
| | gene, Px promoter (U.S. Pat. No. 10,323,261 B2), phaC gene |
| pPYS12 | pBBR1MCS4 derivative (Kovach M E, et al. pBBR1MCS: a |
| | broad-host-range cloning vector. Biotechniques. (1994) 16(5): |
| | 800-802.); BBa_J23119 promoter (The iGEM Parts Registry), 4hbd-2 |
| | gene, crt gene, hbd gene |
| pPYS13 | pBBR1MCS4 derivative (Kovach M E, et al. pBBR1MCS: a |
| | broad-host-range cloning vector. Biotechniques. (1994) 16(5): |
| | 800-802.); BBa_J23119 promoter (The iGEM Parts Registry), 4hbd-3 |
| | gene, crt gene, hbd gene |
| pPYS14 | pBBR1MCS4 derivative (Kovach M E, et al. pBBR1MCS: a |
| | broad-host-range cloning vector. Biotechniques. (1994) 16(5): |
| | 800-802.); BBa J23119 promoter (The iGEM Parts Registry), 4hbd-4 |
| | gene, crt gene, hbd gene |
| pPYS15 | pBBR1MCS4 derivative (Kovach M E, et al. pBBR1MCS: a |
| | broad-host-range cloning vector. Biotechniques. (1994) 16(5): |
| | 800-802.); BBa_J23119 promoter (The iGEM Parts Registry), 4hbd-5 |
| | gene, crt gene, hbd gene |
| pPYS34 | pCL1920 derivative (Lerner C G, Inouye M. Low copy number |
| | plasmids for regulated low-level expression of cloned genes in |
| | Escherichia coli with blue/white insert screening capability. Nucleic |
| | Acids Res. (1990) 18(15): 4631.); PuspA promoter (Prytz et al. |
| | (2003); Dyk et al. (1995); Nyström and Neidhardt (1992)), phaA |
| | gene, Px promoter (U.S. Pat. No. 10,323,261 B2), phaC gene, Px |
| | promoter (U.S. Pat. No. 10,323,261 B2), phaJ gene |
| pPYS35 | pBBR1MCS4 derivative (Kovach M E, et al. pBBR1MCS: a |
| | broad-host-range cloning vector. Biotechniques. (1994) 16(5): |
| | 800-802.); cysK promoter (KR 10-1223904 B1), 4hbd-1 gene, crt |
| | gene, hbd gene |
| pPYS36 | pBBR1MCS4 derivative (Kovach M E, et al. pBBR1MCS: a |
| | broad-host-range cloning vector. Biotechniques. (1994) 16(5): |
| | 800-802.); cysK promoter (KR 10-1223904 B1), 4hbd-2 gene, crt |
| | gene, hbd gene |
| pPYS37 | pBBR1MCS4 derivative (Kovach M E, et al. pBBR1MCS: a |
| | broad-host-range cloning vector. Biotechniques. (1994) 16(5): |
| | 800-802.); cysK promoter (KR 10-1223904 B1), 4hbd-3 gene, crt |
| | gene, hbd gene |
| pPYS38 | pBBR1MCS4 derivative (Kovach M E, et al. pBBR1MCS: a |
| | broad-host-range cloning vector. Biotechniques. (1994) 16(5): |
| | 800-802.); cysK promoter (KR 10-1223904 B1), 4hbd-4 gene, crt |
| | gene, hbd gene |
| pPYS39 | pBBR1MCS4 derivative (Kovach M E, et al. pBBR1MCS: a |
| | broad-host-range cloning vector. Biotechniques. (1994) 16(5): |
| | 800-802.); cysK promoter (KR 10-1223904 B1), 4hbd-5 gene, crt |
| | gene, hbd gene |
| pBBYP1 | pBBR1MCS4 derivative (Kovach M E, et al. pBBR1MCS: a |
| | broad-host-range cloning vector. Biotechniques. (1994) 16(5): |
| | 800-802.); BBa J23119 promoter (The iGEM Parts Registry), phaJ |
| | gene, crt gene, hbd gene |
| pBBYP2 | pBBR1MCS4 derivative (Kovach M E, et al. pBBR1MCS: a |
| | broad-host-range cloning vector. Biotechniques. (1994) 16(5): |
| | 800-802.); BBa_J23119 promoter (The iGEM Parts Registry), 4hbd-1 |
| | gene, crt gene, hbd gene |
| pBBYP3 | pBBR1MCS4 derivative (Kovach M E, et al. pBBR1MCS: a |
| | broad-host-range cloning vector. Biotechniques. (1994) 16(5): |
| | 800-802.); BBa_J23119 promoter (The iGEM Parts Registry), 4hbd-1 |
| | gene, pct gene, Px promoter (U.S. Pat. No. 10,323,261 B2), phaC gene |
| pBBYP4 | pBBR1MCS4 derivative (Kovach M E, et al. pBBR1MCS: a |
| | broad-host-range cloning vector. Biotechniques. (1994) 16(5): |
| | 800-802.); Px promoter (U.S. Pat. No. 10,323,261 B2), phaJ gene, |
| | crt gene, hbd gene |
| pBBYP5 | pBBR1MCS4 derivative (Kovach M E, et al. pBBR1MCS: a |
| | broad-host-range cloning vector. Biotechniques. (1994) 16(5): |
| | 800-802.); BBa_J23119 promoter (The iGEM Parts Registry), phaJ |
| | gene, pct gene, Px promoter (U.S. Pat. No. 10,323,261 B2), phaC gene |

### Example 1: Production of Poly-3-Hydroxybutyrate and Poly-4-Hydroxybutyrate Using Crotonyl-CoA Pathway

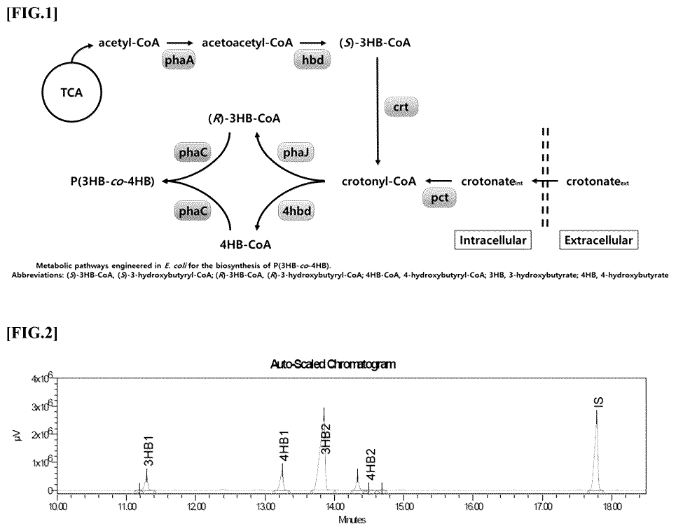

The novel biosynthetic pathway of the poly-3-hydroxybutyrate and poly-4-hydroxybutyrate via crotonyl-CoA is shown in FIG. 1. Crotonyl-CoA is converted to 3-hydroxybutyrate by enoyl-CoA hydratase and is converted to 4-hydroxybutyrate by 4-hydroxybutyryl-CoA dehydratase.

In order to confirm whether 3-hydroxybutyrate and 4-hydroxybutyrate were produced through the novel biosynthetic pathway of the poly-3-hydroxybutyrate and poly-4-hydroxybutyrate via crotonyl-CoA, after adding crotonate from the outside, it was confirmed whether 3-hydroxybutyrate and 4-hydroxybutyrate were produced.

### Example 1-1: Construction of Vector Containing Genes Involved in the Biosynthetic Pathway of Poly-3-Hydroxybutyrate Via Crotonyl-CoA

A recombinant vector pBBYP5, which expresses phaJ (SEQ ID NO: 6), the gene encoding Aeromonas caviae-derived enoyl-CoA hydratase, and pct (SEQ ID NO: 5), the gene encoding Ralstonia eutropha-derived propionyl CoA-transferase, under the expression of BBa_J223119 promoter (The iGEM Parts Registry); and phaC (SEQ ID NO: 2), the gene encoding Ralstonia eutropha-derived PHA synthase, under the expression of Px promoter (U.S. Ser. No. 10/323,261 B2), was constructed using the pBBR1MCS4 vector.

Each gene was amplified by PCR after preparing templates through gene synthesis. The pBBR1MCS4 vector was digested, and PCR was performed using primers at the corresponding recognition sites to insert the amplified gene fragment.

Primer sequences used to construct the recombinant vector are shown in Table 2 below.

TABLE 2

| SEQ ID NO: | Sequence |
|---|---|
| 12 | GAGGGCGGGGTTTTTTTTCT |
| | TCTAGATTGACAGCTAGCTC |
| | AGTCCTAGGTATAATGCTAG |
| | CGTGTTTGCCTGCCCAACAG |
| | AGGAGGACGCCGCATGAGCG |
| | CACAATCCCTGGAAG |
| 13 | CGAATTCCTGCAGCCCGGGG |
| | TTAAGGCAGCTTGACCACGG |
| | C |
| 14 | CCCCGGGCTGCAGGAATTCG |
| | ATATGAAGGTCATTACTGCT |
| | CG |
| 15 | CGAGGTCGACGGTATCGATA |
| | TTATAAGTGCAGGGGCCCAG |
| | C |
| 16 | GCTTATCGATACCGTCGACC |
| | TCGCCAGTCTGGCCTGAACA |
| | TGATATAAAATGAATATAAA |
| | TAGACAGAATAGGGTTATTT |
| | ATGGATTTATATCACTTTAC |
| | AGCCTGTCTCTTGATCAGAT |
| | CTGGCCGCCTAGGCCGAATT |
| | CGAGCTCGGTACCAATTCAG |
| | GAGGTTTTTATGGCGACGGG |
| | AAAAGGTGC |
| 17 | CAAAAGCTGGGTACCGGGCC |
| | CCCCCTCGATCAGGCTTTCG |
| | CTTTTACGTAG |

### Example 1-2: Construction of Vector Containing Genes Involved in the Biosynthetic Pathway of Poly-4-Hydroxybutyrate Via Crotonyl-CoA

A recombinant vector pBBYP3, which includes 4hbd-1 (SEQ ID NO: 7, Konneke, Martin, et al., Proc. Natl. Acad. Sci. USA. 111, 8239-8244, 2014), the gene encoding Nitrosopumilus maritimus-derived 4-hydroxybutyryl-CoA dehydratase; pct (SEQ ID NO: 5), the gene encoding Ralstonia eutropha-derived propionyl CoA-transferase; and phaC (SEQ ID NO: 2), the gene encoding Ralstonia eutropha-derived PHA synthase, under the expression of BBa_J223119 promoter, was constructed using the pBBR1MCS4 vector.

Each gene was amplified by PCR after preparing templates through gene synthesis. The pBBR1MCS4 vector was digested, and PCR was performed using primers at the corresponding recognition sites to insert the amplified gene fragment.

Primer sequences used to construct the recombinant vector are shown in Table 3 below.

TABLE 3

| SEQ ID NO: | Sequence |
|---|---|
| 14 | CCCCGGGCTGCAGGAATTCG |
| | ATATGAAGGTCATTACTGCT |
| | CG |
| 15 | CGAGGTCGACGGTATCGATA |
| | TTATAAGTGCAGGGGCCCAG |
| | C |
| 16 | GCTTATCGATACCGTCGACC |
| | TCGCCAGTCTGGCCTGAACA |
| | TGATATAAAATGAATATAAA |
| | TAGACAGAATAGGGTTATTT |
| | ATGGATTTATATCACTTTAC |
| | AGCCTGTCTCTTGATCAGAT |
| | CTGGCCGCCTAGGCCGAATT |
| | CGAGCTCGGTACCAATTCAG |
| | GAGGTTTTTATGGCGACGGG |
| | AAAAGGTGC |
| 17 | CAAAAGCTGGGTACCGGGCC |
| | CCCCCTCGATCAGGCTTTCG |
| | CTTTTACGTAG |
| 18 | GAGGGCGGGGTTTTTTTTCT |
| | TCTAGATTGACAGCTAGCTC |
| | AGTCCTAGGTATAATGCTAG |
| | CGTGTTTGCCTGCCCAACAG |
| | AGGAGGACGCCGCATGGCGA |
| | ACGTTCTGAAAAC |
| 19 | CGAATTCCTGCAGCCCGGGG |
| | TTACAGCACGGAATCTTTGG |

### Example 1-3: Evaluation of Productivity of Poly-3-Hydroxybutyrate and Poly-4-Hydroxybutyrate of Strains Including Genes Involved in the Biosynthetic Pathway of Poly-3-Hydroxybutyrate or Poly-4-Hydroxybutyrate Via Crotonyl-CoA

In order to confirm whether poly-3-hydroxybutyrate or poly-4-hydroxybutyrate was produced in Escherichia coli through crotonyl-CoA, the recombinant vectors pBBYP5 and pBBYP3 constructed in Examples 1-1 and 1-2 were each transformed into E. coli LS5218 (CGSC strain #6966) to construct recombinant strain containing the genes involved in the biosynthetic pathway of poly-3-hydroxybutyrate or poly-4-hydroxybutyrate via crotonyl-CoA, respectively.

The seed culture was performed as follows: the constructed recombinant strains were cultured under shaking at 37° C. for 16 hours in a 14 mL tube supplemented with 3 mL Luria Bertani (LB) medium (including antibiotics), in the production medium (U.S. Ser. No. 10/323,261 B2). 1.25 mL of the culture solution was inoculated into a 250 mL flask containing a 25 mL production medium containing 50 g/L glucose and cultured at 37° C. at 250 rpm for 5 hours. After adding crotonate (1 g/L) to the culture solution, the strains were cultured under shaking at 250 rpm at 30° C. for 43 hours, and the analysis results by a commonly known GC (gas chromatography) analysis method are shown in Table 4 below.

TABLE 4

| Introduced Gene | 3HB (g/L) | 4HB (g/L) | 3HB or 4HB Content (%) |
|---|---|---|---|
| 4hbd-1, pct, phaC | 0 | 0.27 | 100 |
| phaJ, pct, phaC | 0.66 | 0 | 100 |

As shown in Table 4 above, when phaJ, pct, phaC genes or 4hbd-1, pct, phaC genes were introduced into E. coli, as a result of culturing under the condition of adding 1 g/L of crotonate, it was confirmed that 3-hydroxybutyrate homopolymer (P(3HB)) consisting of 0.66 g/L of 3HB monomer was produced in the recombinant strains introduced with phaJ, pet, phaC genes. Additionally, it was confirmed that 4-hydroxybutyrate homopolymer (P(4HB)) consisting of 0.27 g/L of 4HB monomer was produced in the recombinant strains introduced with 4hbd-1, pct, phaC genes.

### Example 2: Production of Poly-3-Hydroxybutyrate and Poly-4-Hydroxybutyrate Using Crotonyl-CoA Pathway

The novel biosynthetic pathway of the poly-3-hydroxybutyrate and poly-4-hydroxybutyrate via crotonyl-CoA is shown in FIG. 1. Crotonyl-CoA is converted to 3-hydroxybutyrate by enoyl-CoA hydratase and is converted to 4-hydroxybutyrate by 4-hydroxybutyryl-CoA dehydratase. Acetyl-CoA is converted to acetoacetyl-CoA by acetyl-CoA acetyltransferase, acetoacetyl-CoA is converted to (S)-3-hydroxybutyrate-CoA by 3-hydroxybutyryl-CoA dehydrogenase, (S)-3-hydroxybutyrate-CoA is converted to crotonyl-CoA by 3-hydroxybutyryl-CoA dehydratase. Subsequently, crotonyl-CoA is converted to poly-3-hydroxybutyrate by enoyl-CoA hydratase and PHA synthase, and is converted to poly-4-hydroxybutyrate by 4-hydroxybutyryl-CoA dehydratase and PHA synthase.

In order to confirm whether 3-hydroxybutyrate and 4-hydroxybutyrate were produced through the novel biosynthetic pathway of the poly-3-hydroxybutyrate and poly-4-hydroxybutyrate via crotonyl-CoA, after introducing each foreign enzyme required for the novel biosynthetic pathway into E. coli, it was confirmed whether poly-3-hydroxybutyrate and poly-4-hydroxybutyrate were produced.

### Example 2-1: Construction of Vector Containing Genes Involved in the Biosynthetic PathwayBiosynthetic Pathway of Poly-3-Hydroxybutyrate Via Crotonyl-CoA

A recombinant vector pPYS11 was constructed by digesting the recombinant vector pPYS10 with XbaI, performing PCR using primers of SEQ ID NOS: 22 and 23 at the corresponding recognition sites, and inserting the amplified PuspA_phaA DNA fragment, such that phaA (SEQ ID NO: 1), the gene encoding Ralstonia eutropha-derived acetyl-CoA acetyltransferase, could be expressed under the PuspA (universal stress protein A promoter) (Prytz et al. 2003; Dyk et al. 1995; Nystrom and Neidhardt 1992; 1994) promoter, and phaC (SEQ ID NO: 2), the gene encoding Ralstonia eutropha-derived PHA synthase, could be expressed under the Px promoter (U.S. Ser. No. 10/323,261 B2).

Meanwhile, a recombinant vector pBBYP1, which includes hbd (SEQ ID NO: 4), the gene encoding Clostridium acetobutylicum-derived 3-hydroxybutyryl-CoA dehydrogenase; crt (SEQ ID NO: 3), the gene encoding Clostridium acetobutylicum-derived 3-hydroxybutyryl-CoA dehydrogenase; and phaJ (SEQ ID NO: 6), the gene encoding Aeromonas caviae-derived enoyl-CoA hydratase, under the expression of BioBrick BBa_J23119 promoter, was constructed using the pBBR1MCS4 vector.

Primer sequences used to construct the recombinant vector are shown in Table 5 below.

TABLE 5

| SEQ ID NO: | Sequence |
|---|---|
| 22 | ACAGAAGGCTTAAGGATCCT |
| | CGGTTTCTTCAGAGATTTAA |
| | AACCACTATCAATATATTCA |
| | TGTCGAAAATTTG |
| 23 | TGCATGCCTGCAGGTCGACT |
| | TTATTTGCGCTCAACTGCTA |
| | AC |

### Example 2-2: Construction of Vector Containing Genes Involved in the Biosynthetic Pathway of Poly-4-Hydroxybutyrate Via Crotonyl-CoA

A recombinant vector pPYS11 was constructed by digesting the recombinant vector pPYS10 with XbaI, performing PCR using primers of SEQ ID NOS: 22 and 23 at the corresponding recognition sites, and inserting the amplified PuspA_phaA DNA fragment, such that phaA (SEQ ID NO: 1), the gene encoding Ralstonia eutropha-derived acetyl-CoA acetyltransferase, could be expressed under the PuspA (universal stress protein A promoter) (Prytz et al. 2003; Dyk et al. 1995; Nyström and Neidhardt 1992; 1994) promoter, and phaC (SEQ ID NO: 2), the gene encoding Ralstonia eutropha-derived PHA synthase, could be expressed under the Px promoter (U.S. Ser. No. 10/323,261 B2).

Meanwhile, recombinant vectors containing any one of the genes 4hbd-1 to 4hbd-5 encoding 4-hydroxybutyryl-CoA dehydratase were respectively prepared. Specifically, a recombinant vector pBBYP2 including Nitrosopumilus maritimus-derived 4hbd-1 (SEQ ID NO: 7) was constructed, and 4 types of recombinant vectors (pPYS12/pPYS13/pPYS14/pPYS15), in which Candidatus Nitrosopelagicus brevis-derived 4hbd-2 (SEQ ID NO: 8), Candidatus Nitrosopelagicus brevis-derived 4hbd-3 (SEQ ID NO: 9), Candidatus Nitrosopelagicus limnium-derived 4hbd-4 (SEQ ID NO: 10), and Thaumarchaeota archaeon-derived 4hbd-5 (SEQ ID NO: 11) were inserted instead of 4hbd-1 under the BBa_J23119 promoter, were constructed based on pBBYP2, respectively. More specifically, pPYS12 was constructed by digesting the previously-owned recombinant vector pBBYP2, performing PCR using primers of SEQ ID NOS: 24 and 25 at the corresponding recognition sites, and inserting the amplified 4hbd-2 DNA fragment. In addition, pPYS13 was constructed by performing PCR using primers of SEQ ID NOS: 26 and 27 at the corresponding recognition sites, and inserting the amplified 4hbd-3 DNA fragment. Further, pPYS14 was constructed by performing PCR using primers of SEQ ID NOS: 28 and 29 at the corresponding recognition sites, and inserting the amplified 4hbd-4 DNA fragment. Moreover, pPYS15 was constructed by performing PCR using primers of SEQ ID NOS: 30 and 31 at the corresponding recognition sites, and inserting the amplified 4hbd-5 DNA fragment.

Primer sequences used to construct the recombinant vector are shown in Table 6 below.

TABLE 6

| SEQ ID NO: | Sequence |
|---|---|
| 22 | ACAGAAGGCTTAAGGATCCT |
| | CGGTTTCTTCAGAGATTTAA |
| | AACCACTATCAATATATTCA |
| | TGTCGAAAATTTG |
| 23 | TGCATGCCTGCAGGTCGACT |
| | TTATTTGCGCTCAACTGCTA |
| | AC |
| 24 | GAGGGCGGGGTTTTTTTTCT |
| | TTTGACAGCTAGCTCAGTCC |
| | TAGGTATAATGCTAGCTTAA |
| | TTAATCTAGGGTACCAGGAG |
| | GTTTTTATGCCTATCAAGAA |
| | TGGCGC |
| 25 | GTTCCATAAAAACCTCCTCC |
| | CCTATTTCACCTTTTTCTTA |
| | TCCTTGG |
| 26 | GAGGGCGGGGTTTTTTTTCT |
| | TTTGACAGCTAGCTCAGTCC |
| | TAGGTATAATGCTAGCTTAA |
| | TTAATCTAGGGTACCAGGAG |
| | GTTTTTATGCAGAAAACTGT |
| | CAAACC |
| 27 | GTTCCATAAAAACCTCCTCC |
| | CTTACTTTTCAGGGATCTTA |
| | AAAAC |
| 28 | GAGGGCGGGGTTTTTTTTCT |
| | TTTGACAGCTAGCTCAGTCC |
| | TAGGTATAATGCTAGCTTAA |
| | TTAATCTAGGGTACCAGGAG |
| | GTTTTTATGGCAAACGTACT |
| | GAAAAC |
| 29 | GTTCCATAAAAACCTCCTCC |
| | CCTATAAAACCGAATCTTTG |
| | GTC |
| 30 | GAGGGCGGGGTTTTTTTTCT |
| | TTTGACAGCTAGCTCAGTCC |
| | TAGGTATAATGCTAGCTTAA |
| | TTAATCTAGGGTACCAGGAG |
| | GTTTTTATGCAAAAAACCGT |
| | GCGCCC |
| 31 | GTTCCATAAAAACCTCCTCC |
| | CTTACTCTTTAGAGTCGTCC |
| | AG |

### Example 2-3: Evaluation of Productivity of Poly-3-Hydroxybutyrate and Poly-4-Hydroxybutyrate of Strains Including Genes Involved in the Biosynthetic pathwayBiosynthetic Pathway of Poly-3-Hydroxybutyrate or Poly-4-Hydroxybutyrate Via Crotonyl-CoA

In order to confirm whether poly-3-hydroxybutyrate was produced through crotonyl-CoA in E. coli, the recombinant vector pPYS11 constructed in Example 2-1 was transformed into E. coli LS5218 (CGSC strain #6966) strain, and then the recombinant vector pBBYP1 constructed in Example 2-1 was further transformed to construct a P(3HB)-producing recombinant strain including the genes involved in the biosynthetic pathway of the poly-3-hydroxybutyrate via crotonyl-CoA.

Meanwhile, in order to confirm whether poly-4-hydroxybutyrate was produced through crotonyl-CoA in E. coli, the recombinant vector pPYS11 constructed in Example 2-2 was transformed into E. coli LS5218 (CGSC strain #6966) strain, and then the 5 types of recombinant vectors (pBBYP2/pPYS12/pPYS13/pPYS14/pPYS15) constructed in Example 2-2 were further transformed to construct 5 types of P(4HB)-producing recombinant strains including the genes involved in the biosynthetic pathway of the poly-4-hydroxybutyrate via crotonyl-CoA, respectively.

The seed culture of one type of P(3HB)-producing recombinant strain and 5 types of P(4HB)-producing recombinant strains was carried out as follows: the strains were cultured with shaking overnight at 37° C. in a 14 mL tube supplemented with 3 mL LB medium (including antibiotics), and 1.25 mL of the culture solution was inoculated into a 250 mL flask containing a 25 mL production medium (U.S. Ser. No. 10/323,261 B2) containing 50 g/L glucose and cultured at 37° C. at 250 rpm for 5 hours, and then cultured under shaking for a total of 48 hours by lowering the temperature to 30° C. The composition of the production medium is as follows.

After completion of the culture, the analysis results by a commonly known GC (gas chromatography) analysis method are shown in Table 7 below.

TABLE 7

| Common Gene | Single Gene | 3HB (g/L) | 4HB (g/L) |
|---|---|---|---|
| phaA, phaC, | phaJ | 5.1 | 0 |
| crt, hbd | 4hbd-1 | 0 | 0.06 |
| | 4hbd-2 | | 0.10 |
| | 4hbd-3 | | 0.14 |
| | 4hbd-4 | | 0.12 |
| | 4hbd-5 | | 0.07 |

As shown in Table 7, it was confirmed that P(3HB) homopolymer containing 5.1 g/L of 3HB was produced in the P(3HB)-producing recombinant strain. It was confirmed that P(4HB) homopolymers containing different amounts of 4HB were produced in the 5 types of P(4HB)-producing recombinant strains depending on the type of 4hbd gene. Specifically, it was confirmed that 0.06 g/L of 4HB was produced in the recombinant strain expressing 4hbd-1, 0.10 g/L of 4HB was produced in the recombinant strain expressing 4hbd-2, 0.14 g/L of 4HB was produced in the recombinant strain expressing 4hbd-3, 0.12 g/L of 4HB was produced in the recombinant strain expressing 4hbd-4, and P(4HB) homopolymer containing 0.07 g/L of 4HB monomer was produced in the recombinant strain expressing 4hbd-5, respectively.

### Example 3: Production of Poly(3-Hydroxybutyrate-Co-4-Hydroxybutyrate) Using Crotonyl-CoA Pathway

The novel biosynthetic pathway of the poly(3-hydroxybutyrate-co-4-hydroxybutyrate) via crotonyl-CoA is shown in FIG. 1.

In order to confirm whether poly(3-hydroxybutyrate-co-4-hydroxybutyrate) was produced through the novel biosynthetic pathway of the poly(3-hydroxybutyrate-co-4-hydroxybutyrate) via crotonyl-CoA, after introducing each foreign enzyme required for the novel biosynthetic pathway into E. coli, it was confirmed whether poly(3-hydroxybutyrate-co-4-hydroxybutyrate) was produced.

### Example 3-1: Construction of Vector Containing Genes Involved in the Biosynthetic pathwayBiosynthetic Pathway of Poly(3-Hydroxybutyrate-Co-4-Hydroxybutyrate) Via Crotonyl-CoA

As shown in Example 2-3, in the production of 3HB and 4HB of the P(3HB)-producing recombinant strain and the P(4HB)-producing recombinant strains, the production of 4HB is relatively very low. Accordingly, it was attempted to produce poly(3-hydroxybutyrate-co-4-hydroxybutyrate) having various 4-hydroxybutyrate contents by relatively increasing the production of 4HB monomer and decreasing the production of 3HB monomer.

To this end, it was attempted to lower the metabolic flux of phaJ, the gene encoding an enzyme that converts crotonyl-CoA into 3-hydroxybutyryl-CoA, by regulating the intensity of the promoter.

Specifically, based on the recombinant vector pBBYP1 constructed in Example 2-1, a recombinant vector pBBYP4 with weakened phaJ expression level was constructed using the Px promoter (U.S. Ser. No. 10/323,261 B2), which is about 80% of the strength of the BBa_J23119 promoter used for phaJ gene expression. Specifically, the recombinant vector pBBYP1 was digested with XbaI and SmaI, and PCR was performed based on pPYS34 as a template using the primers of SEQ ID NOS: 32 and 33 to insert the amplified Px_phaJ DNA fragment into the corresponding recognition site.

Primer sequences used to construct the recombinant vectors are shown in Table 8 below.

TABLE 8

| SEQ ID NO: | Sequence |
|---|---|
| 32 | GAGGGCGGGGTTTTTTTTCT |
| | TCTAGTCGCCAGTCTGGCCT |
| | GAACATG |
| 33 | TTAAGGCAGCTTGACCACGG |
| | GTTCCATAAAAACCTCCTCC |
| | C |

### Example 3-2: Evaluation of Productivity of Poly(3-Hydroxybutyrate-Co-4-Hydroxybutyrate) of Strains Including Genes Involved in the Biosynthetic Pathway of Poly(3-Hydroxybutyrate-Co-4-Hydroxybutyrate) Via Crotonyl-CoA

In order to confirm whether poly(3-hydroxybutyrate-co-4-hydroxybutyrate) having various 4-hydroxybutyrate contents was produced through crotonyl-CoA in E. coli, the recombinant vector pPYS11 constructed in Example 2-1 was transformed into E. coli LS5218 (CGSC strain #6966) strain, and the recombinant vector pBBYP4 constructed in Example 3-1 was further transformed thereinto, and then the 5 types of recombinant vectors (pBBYP2/pPYS12/pPYS13/pPYS14/pPYS15) constructed in Example 2-2 were each transformed to construct a Poly(3-hydroxybutyrate-co-4-hydroxybutyrate)-producing recombinant strain including the genes involved in the biosynthetic pathway of the poly(3-hydroxybutyrate-co-4-hydroxybutyrate) via crotonyl-CoA.

The constructed Poly(3-hydroxybutyrate-co-4-hydroxybutyrate)-producing recombinant strain was cultured under the same conditions as in Example 2-3.

After the completion of the culture, the monomer composition and content of the constructed Poly(3-hydroxybutyrate-co-4-hydroxybutyrate) were analyzed by GC (gas chromatography) analysis.

Specifically, the cells collected after culturing were washed with distilled water, and then the cells were dried using a freeze dryer. In order to extract PHA from freeze-dried cells, a reaction solution, which was prepared by mixing 250 ml of n-butanol, 4M hydrochloric acid dissolved in 250 ml of dioxene, and 1 g of diphenylmethane, was added to freeze-dried cells DCW (dry cell weight). Then, the cells were subjected to sonication at 70° C. or higher. After confirming that the sample was completely dissolved, it was sufficiently reacted at 95° C., and distilled water was added to separate the layers, and then the supernatant was collected to prepare a sample for analysis. Subsequently, the content and composition of poly(3-hydroxybutyrate-co-4-hydroxybutyrate) was analyzed using the prepared sample through a gas chromatography-flame ionization detector (GC-FID, Shimadzu GC2010plus). For GC-FID analysis, a DB-FFAP (30 m, 0.25 mm, 0.25 μm) capillary column was installed. The split ratio was 1/10, helium was used as the mobile phase, and the inlet and detector temperatures were set to 200° C. and 230° C., respectively. The oven temperature was initially at 80° C. (maintained for 5 minutes) and then raised to 220° C. at a rate of 10° C./min.

The analysis results are shown in Table 9 below.

TABLE 9

| Common Gene | Single Gene | 3HB (g/L) | 4HB (g/L) | 4HB Content (%) |
|---|---|---|---|---|
| phaA, hbd, | 4hbd-1 | 3.32 | 0.029 | 0.87 |
| crt, phaJ, | 4hbd-2 | 3.05 | 0.016 | 0.52 |
| phaC | 4hbd-3 | 2.89 | 0.058 | 1.97 |
| | 4hbd-4 | 3.11 | 0.025 | 0.80 |
| | 4hbd-5 | 3.89 | 0.031 | 0.79 |

As shown in Table 9, as a result of analyzing the 3HB and 4HB monomer contents in the 5 types of Poly(3-hydroxybutyrate-co-4-hydroxybutyrate)-producing recombinant strains containing each of 4hbd-1/4hbd-2/4hbd-3/4hbd-4/4hbd-5 genes, respectively, it was confirmed that poly(3-hydroxybutyrate-co-4-hydroxybutyrate) having various 4-hydroxybutyrate contents were produced. It was confirmed that 3.32 g/L of 3HB and 0.029 g/L of 4HB were produced in the Poly(3-hydroxybutyrate-co-4-hydroxybutyrate)-producing recombinant strain containing the 4hbd-1 gene, and the 4HB content was about 0.87%. In addition, it was confirmed that the Poly(3-hydroxybutyrate-co-4-hydroxybutyrate)-producing recombinant strain in which the 4hbd-3 gene was expressed could produce Poly(3-hydroxybutyrate-co-4-hydroxybutyrate) copolymer having the highest 4HB content of about 1.97%.

### Example 4: Production of Poly(3-Hydroxybutyrate-Co-4-Hydroxybutyrate) Using Crotonyl-CoA Pathway

### Example 4-1: Construction of Vector Containing Genes Involved in the Biosynthetic pathwayBiosynthetic Pathway of Poly(3-Hydroxybutyrate-Co-4-Hydroxybutyrate) Via Crotonyl-CoA

As shown in Example 2-3, in the production of 3HB and 4HB of the P(3HB)-producing recombinant strain and the P(4HB)-producing recombinant strains, the production of 4HB is relatively very low. Accordingly, it was attempted to produce poly(3-hydroxybutyrate-co-4-hydroxybutyrate) having various 4-hydroxybutyrate contents by relatively increasing the production of 4HB monomer and decreasing the production of 3HB monomer.

To this end, it was attempted to regulate the metabolic flux of the 3HB and 4HB pathways by reducing the copy number of the plasmid expressing the phaJ gene of the 3HB pathway and enhancing the promoter expressing the 4hbd gene of the 4HB pathway.

Specifically, it was attempted to express the phaJ gene in the plasmid pCL1920 (about 5 copy number, Lerner & Inouye, 1990, NAR 18, 15 p. 4631) having a lower copy number than the recombinant vector pBBYP4 (about 15 to 20 copy number) constructed in Example 3-1. Specifically, a new recombinant vector pPYS34 was constructed based on the recombinant vector pPYS11 based on the pCL1920 vector constructed in Example 2-1. The pPYS34 was constructed by digesting pPYS11 with HindIII, and performing PCR using the primers of SEQ ID NOS: 34 and 35 at the corresponding recognition sites to insert the DNA fragment with the Px promoter (U.S. Pat. No. 10,323,261 B2) attached to the phaJ gene amplified from pBBYP1.

Primer sequences used to construct the recombinant vector are shown in Table 10 below.

TABLE 10

| SEQ ID NO: | Sequence |
|---|---|
| 34 | AGTCGACCTGCAGGCATGCA |
| | TCGCCAGTCTGGCCTGAACA |
| | TGATATAAAATGTGTTTGCC |
| | TGCCCAACAGA |
| 35 | CTATGACCATGATTACGCCA |
| | TTAAGGCAGCTTGACCACGG |

Meanwhile, in order to increase the metabolic flux of the 4HB pathway, it was attempted to enhance the gene expression promoter to increase the expression of the 4hbd gene in the 4HB pathway. Specifically, 5 types of new recombinant vectors (pPYS35/pPYS36/pPYS37/pPYS38/pPYS39) expressing a stronger PcysK promoter instead of the BBa_J23119 promoter were prepared based on the 5 types of the recombinant vectors (pBBYP2/pPYS12/pPYS13/pPYS14/pPYS15) constructed under the BBa_J23119 promoter in Example 2-2. Specifically, in the case of pPYS35 construction, the previously owned recombinant vector pBBYP2 was digested with XbaI and PacI, and the PcysK DNA fragment amplified with the primers of SEQ ID NOS: 36 and 37 was inserted into the corresponding recognition site. In the case of pPYS36 construction, the previously owned recombinant vector pPYS12 was digested with PacI, and the PcysK DNA fragment amplified with the primers of SEQ ID NOS: 38 and 39 was inserted into the corresponding recognition site. In the case of pPYS37/pPYS38/pPYS39 construction, the pPYS13/pPYS14/pPYS15 vectors were also digested with PacI, and the PcysK DNA fragment amplified with the primers of SEQ ID NOS: 38 and 39 was inserted into the corresponding recognition site.

Primer sequences used to construct the recombinant vectors are shown in Table 11 below.

TABLE 11

| SEQ ID NO: | Sequence |
|---|---|
| 36 | AGGGCGGGGTTTTTTTTCTT |
| | CCAGCCTGTTTACGATGATC |
| 37 | CTGGTACCCTAGATTAATTA |
| | ATCCTTAACTGTATGAAATT |
| | GGG |
| 38 | CTAGGTATAATGCTAGCTTA |
| | ATCCAGCCTGTTTACGATGA |
| | TC |
| 39 | CTCCTGGTACCCTAGATTAA |
| | TTCCTTAACTGTATGAAATT |
| | GGG |

### Example 4-2: Evaluation of Productivity of Poly(3-Hydroxybutyrate-Co-4-Hydroxybutyrate) of Strains Including Genes Involved in the Biosynthetic Pathway of Poly(3-Hydroxybutyrate-Co-4-Hydroxybutyrate Via Crotonyl-CoA

In order to confirm whether poly(3-hydroxybutyrate-co-4-hydroxybutyrate) having various 4-hydroxybutyrate contents was produced through crotonyl-CoA in E. coli, the recombinant vector pPYS11 constructed in Example 2-1 was transformed into E. coli LS5218 (CGSC strain #6966) strain, and the recombinant vector pPYS34 constructed in Example 4-1 was further transformed thereinto, and then the 5 types of recombinant vectors (pPYS35/pPYS36/pPYS37/pPYS38/pPYS39) constructed in Example 4-1 were each transformed to construct a Poly(3-hydroxybutyrate-co-4-hydroxybutyrate)-producing recombinant strain including the genes involved in the biosynthetic pathway of the poly(3-hydroxybutyrate-co-4-hydroxybutyrate) via crotonyl-CoA.

The constructed Poly(3-hydroxybutyrate-co-4-hydroxybutyrate)-producing recombinant strain was cultured under the same conditions as in Example 2-3.

After completion of the culture, the results analyzed by GC (gas chromatography) analysis in the same manner as in Example 3-2 are shown in Table 12 below.

TABLE 12

| Common Gene | Single Gene | 3HB (g/L) | 4HB (g/L) | 4HB Content (%) |
|---|---|---|---|---|
| phaA, hbd, | 4hbd-1 | 2.32 | 0.112 | 4.61 |
| crt, phaJ, | 4hbd-2 | 1.98 | 0.096 | 4.62 |
| phaC | 4hbd-3 | 1.77 | 0.135 | 7.09 |
| | 4hbd-4 | 2.28 | 0.072 | 3.06 |
| | 4hbd-5 | 3.11 | 0.065 | 2.05 |

As shown in Table 12, it was confirmed that poly(3-hydroxybutyrate-co-4-hydroxybutyrate) having various 4-hydroxybutyrate contents were produced by regulating the expression level of the phaJ gene and the expression level of the 4hbd gene. Specifically, it was confirmed that the Poly(3-hydroxybutyrate-co-4-hydroxybutyrate) copolymer containing more 4HB monomeric monomers than Example 3-2 was produced. In addition, it was confirmed that the Poly(3-hydroxybutyrate-co-4-hydroxybutyrate)-producing strain in which the 4hbd-3 gene was expressed could product the Poly(3-hydroxybutyrate-co-4-hydroxybutyrate) copolymer having the highest 4HB content of about 7.09%.

### Example 5: Production of Poly(3-Hydroxybutyrate-Co-4-Hydroxybutyrate) Using Crotonyl-CoA Pathway

### Example 5-1: Construction of Vector Containing Genes Involved in the Biosynthetic Pathway of Poly(3-Hydroxybutyrate-Co-4-Hydroxybutyrate) Via Crotonyl-CoA

As shown in Example 2-3, in the production of 3HB and 4HB of the P(3HB)-producing recombinant strain and the P(4HB)-producing recombinant strains, the production of 4HB is relatively very low. Accordingly, it was attempted to produce poly(3-hydroxybutyrate-co-4-hydroxybutyrate) having various 4-hydroxybutyrate contents by relatively increasing the production of 4HB monomer and decreasing the production of 3HB monomer.

To this end, it was attempted to regulate the 4HB content of the copolymer by inserting the phaJ gene of the 3HB pathway into the genome of recombinant E. coli, thereby relatively decreasing the expression level of the 3HB biosynthetic gene compared to the expression of the pPYS34 recombinant vector based on the pCL1920 vector used in Example 4-1.

Specifically, a recombinant strain, in which the phaJ gene was inserted into the genome, was constructed using a two-step homologous recombination process (Blomfield, I. C., et al., 1991) in order to decrease the expression copy number of the phaJ gene to 1.

More specifically, the chromosomal gene maeB of the E. coli LS5218 (CGSC strain #6966) strain (US 2020/0048642 A1) was removed and the phaJ gene was inserted in its place. In order to prepare for genomic insertion, a reverse-selected suicide vector pSKYP2 consisting of the levansucrase gene (sacB), the phaJ gene under the Px promoter (U.S. Ser. No. 10/323,261 B2), and the kanamycin antibiotic selectable marker was constructed. The use of the sacB vector for gene replacement is also described on the website (arep.med.harvard.edu/labgc/pko3.html). Prior to construction of the pSKYP2 plasmid, pSKH130 was digested with the restriction enzyme EcoRV to prepare the gene replacement vector pSKYP1 containing the sacB gene and the R6K origin. The reaction mixture of the PCR mixture and EcoRV digestion was purified with a QIAGEN purification kit and then eluted to obtain a first 0.5 kb DNA fragment, a second 0.5 kb DNA fragment, and a 4.7 kb vector DNA fragment.

The constructed plasmid pSKYP1 was digested with SalI, PCR was performed with the primers of SEQ ID NOS: 40 and 41 at the corresponding recognition site, and the DNA fragment with the Px promoter (U.S. Ser. No. 10/323,261 B2) attached to the phaJ gene amplified from pPYS34 was inserted to construct the pSKYP2 recombinant vector. In order to replace maeB with phaJ on the chromosome of Escherichia co/i LS5218, the pSKYP2 plasmid was introduced into E. coli strain LS5218, the pSKYP2 plasmid was introduced into the E. coli strain LS5218 by electroporation, and then single colonies that grew on LB agar plates containing 50 mg/L of kanamycin (Km) were selected. Thereafter, the chromosomal insertion of the selected colonies was confirmed by PCR, and in order to “pop out” the sacB gene and the R6K origin in the selected strains, the colonies were grown on LB agar plates without NaCl but containing 20% sucrose for 16 hours. Transformants were used to confirm the replacement of LS5218 maeB by phaJ using PCR and sequence verification. The resulting strain with the correct genotype was designated as E. coli YP1 (E. coli LS5218 ΔmaeB:: Px promoter-phaJ).

Primer sequences used are shown in Table 13 below.

TABLE 13

| SEQ ID NO: | Sequence |
|---|---|
| 40 | GTTACGTGAAAGGAACAACC |
| | AAGTCGATCGCCAGTCTGGC |
| | CTGAACATG |
| 41 | TAAGCGTGAGAGTTAAAAAA |
| | AAGTTAAGGCAGCTTGACCA |
| | CGG |

### Example 5-2: Evaluation of Productivity of Poly(3-Hydroxybutyrate-Co-4-Hydroxybutyrate) of Strains Including Genes Involved in the Biosynthetic pathwayBiosynthetic Pathway of Poly(3-Hydroxybutyrate-Co-4-Hydroxybutyrate) Via Crotonyl-CoA

In order to confirm whether poly(3-hydroxybutyrate-co-4-hydroxybutyrate) having various 4-hydroxybutyrate contents was produced through crotonyl-CoA in E. coli, the recombinant vector pPYS11 constructed in Example 2-1 was transformed into E. coli YP1 strain constructed in Example 5-1, and then the 5 types of recombinant vectors (pPYS35/pPYS36/pPYS37/pPYS38/pPYS39) constructed in Example 4-1 were each transformed to construct a Poly(3-hydroxybutyrate-co-4-hydroxybutyrate)-producing recombinant strain including the genes involved in the biosynthetic pathway of the poly(3-hydroxybutyrate-co-4-hydroxybutyrate) via crotonyl-CoA.

The constructed Poly(3-hydroxybutyrate-co-4-hydroxybutyrate)-producing recombinant strain was cultured under the same conditions as in Example 2-3.

After completion of the culture, the results analyzed by GC (gas chromatography) analysis in the same manner as in Example 3-2 are shown in FIG. 2 and Table 14 below. IS in FIG. 2 means internal standard

TABLE 14

| Common Gene | Single Gene | 3HB (g/L) | 4HB (g/L) | 4HB Content (%) |
|---|---|---|---|---|
| phaA, hbd, | 4hbd-1 | 2.01 | 0.126 | 5.90 |
| crt, phaJ, | 4hbd-2 | 1.55 | 0.113 | 6.79 |
| phaC | 4hbd-3 | 1.38 | 0.192 | 12.21 |
| | 4hbd-4 | 1.72 | 0.154 | 8.22 |
| | 4hbd-5 | 2.10 | 0.087 | 3.98 |

As shown in FIG. 2 and Table 14, it was confirmed that poly(3-hydroxybutyrate-co-4-hydroxybutyrate) with various 4-hydroxybutyrate contents were produced by regulating the 3HB metabolic flux through genome insertion of the phaJ gene. Specifically, it was confirmed that the Poly(3-hydroxybutyrate-co-4-hydroxybutyrate) copolymer containing more 4HB monomeric monomers than Example 4-2 was produced. In addition, it was confirmed that the Poly(3-hydroxybutyrate-co-4-hydroxybutyrate)-producing recombinant strain in which the 4hbd-3 gene was expressed could produce the Poly(3-hydroxybutyrate-co-4-hydroxybutyrate) copolymer having the highest 4HB content of about 12.21%.

From the foregoing, a skilled person in the art to which the present disclosure pertains will be able to understand that the present disclosure may be embodied in other specific forms without modifying the technical concepts or essential characteristics of the present disclosure. In this regard, the exemplary embodiments disclosed herein are only for illustrative purposes and should not be construed as limiting the scope of the present disclosure. The scope of the present disclosure is therefore indicated by the appended claims rather than by the foregoing description. All changes which come within the meaning and range of equivalency of the claims are to be embraced within the scope of the present disclosure.

1. A method for preparing poly(3-hydroxybutyrate-co-4-hydroxybutyrate), comprising:
(a) converting crotonyl-CoA into 3-hydroxybutyryl-CoA in the presence of enoyl-CoA hydratase and converting crotonyl-CoA into 4-hydroxybutyryl-CoA in the presence of 4-hydroxybutyryl-CoA dehydratase; and
(b) preparing poly(3-hydroxybutyrate-co-4-hydroxybutyrate) by polymerization of 3-hydroxybutyryl-CoA and 4-hydroxybutyryl-CoA in the presence of PHA synthase.

2. The method of claim 1, wherein the method further comprises the following steps (i) to (iii), or step (iv), prior to step (a) above:
(i) converting acetyl-CoA into acetoacetyl-CoA in the presence of CoA acetyltransferase;
(ii) converting acetoacetyl-CoA into 3-hydroxybutyryl-CoA in the presence of 3-hydroxybutyryl-CoA dehydrogenase; and
(iii) converting 3-hydroxybutyryl-CoA into crotonyl-CoA using 3HB-CoA dehydratase; or
(iv) converting crotonate into crotonyl-CoA in the presence of propionyl CoA-transferase.

3. The method of claim 1 wherein the poly(3-hydroxybutyrate-co-4-hydroxybutyrate) has a 4-hydroxybutyrate content of 0.1 wt % to 60 wt %.

4. The method according to claim 14, wherein the microorganism comprises a gene encoding enoyl-CoA hydratase, a gene encoding 4-hydroxybutyryl-CoA dehydratase, and a gene encoding PHA synthase.

5. The method of claim 4, wherein the microorganism is a microorganism of the genus Escherichia.

6. The method of claim 5, wherein the microorganism of the genus Escherichia is Escherichia coli.

7. The method of claim 4, wherein the microorganism further comprises: (i′) a gene encoding acetyl-CoA acetyltransferase, a gene encoding 3-hydroxybutyryl-CoA dehydrogenase, and a gene encoding 3HB-CoA dehydratase; or
(ii′) a gene encoding propionyl CoA-transferase.

8. The method of claim 4, wherein an activity of enoyl-CoA hydratase and an activity of 4-hydroxybutyryl-CoA dehydratase is regulated.

9. The method of claim 8, wherein the poly(3-hydroxybutyrate-co-4-hydroxybutyrate) has a 4-hydroxybutyrate content of 0.1 wt % to 60 wt %.

10-12. (canceled)

13. The method according to claim 1, wherein (i) the enoyl-CoA hydratase and the 4-hydroxybutyryl-CoA dehydratase, and (ii) the PHA synthase are produced by a microorganism.

14. The method according to claim 1, further comprising producing (i) the enoyl-CoA hydratase and the 4-hydroxybutyryl-CoA dehydratase, and (ii) the PHA synthase by culturing a microorganism.

15. The method according to claim 14, wherein the microorganism is a prokaryotic organism.

16. The method according to claim 14, wherein the microorganism is a eukaryotic microorganism.

17. The method according to claim 14, wherein the microorganism belongs to the genus Escherichia.

18. The method according to claim 14, wherein the microorganism belongs to the genus Erwinia.

19. The method according to claim 14, wherein the microorganism belongs to the genus Serratia.

20. The method according to claim 14, wherein the microorganism belongs to the genus Providencia.

21. The method according to claim 14, wherein the microorganism belongs to the genus Corynebacterium,

22. The method according to claim 14, wherein the microorganism belongs to the genus Pseudomonas.

23. The method according to claim 14, wherein the microorganism is yeast.


SEQUENCE LISTING (41 sequences)

SEQ ID NO: 1 - DNA, 1182 bp, Ralstonia sp.
  source 1..1182: mol_type = genomic DNA
atgactgatgtggtgatcgtctctgcagctcgcaccgcagtgggcaagttcgggggatctcttgccaaaattccggctcccgagttgggtgccgtcgtcatcaaggccgcattggaacgcgcaggggtgaagcccgaacaagtatcagaggtgattatgggccaagtgttaacagcgggctctgggcagaacccagcacgtcaagccgctattaaagccggtctgcccgcgatggtgccagccatgacgatcaacaaagtttgtgggtcagggttgaaagcggtcatgcttgcggccaacgcaatcatggcaggggatgctgaaatcgtggtggcggggggtcaagagaatatgtccgcagccccgcatgtgcttcctggatcgcgtgatggctttcgtatgggcgatgccaagctggtggatacgatgatcgttgacgggctgtgggacgtgtacaaccaataccacatgggtatcacggcagagaacgttgctaaggaatacggtatcacgcgtgaagcacaggatgaatttgcagtgggatcacagaataaagcagaggcggcgcagaaggctgggaagttcgacgaggaaatcgttcccgtacttatccctcagcgcaaaggtgaccctgtcgcattcaagaccgatgagtttgttcgtcagggggctacattagacagtatgtccggtctgaaacctgcgttcgacaaggccgggacggtcactgccgcgaacgcgtcgggcttgaacgatggggcagcagctgtggtagtcatgtcagcagccaaggccaaagaattaggtttgactcccttagccacaattaaatcatacgctaacgcaggtgtcgacccgaaagtaatgggtatgggacccgttcctgcatcgaaacgcgcgctttctcgtgccgagtggacacctcaagatttagacctgatggagatcaacgaggcttttgctgcccaagctcttgctgtgcatcagcagatgggttgggacacaagtaaggtaaatgttaacgggggcgcaattgcgattgggcaccctattggtgcgtccggctgtcgtatcttggtgacccttttacatgaaatgaaacgtcgcgatgctaaaaaaggtctggcgtcgctgtgtatcggaggtggaatgggcgtcgcgttagcagttgagcgcaaataa

SEQ ID NO: 2 - DNA, 1770 bp, Ralstonia sp.
  source 1..1770: mol_type = genomic DNA
atggcgacgggaaaaggtgctgctgctagcacccaggagggaaagtctcaacccttcaaagtaacgcctggacccttcgacccggctacctggcttgagtggtcacgtcaatggcaaggtaccgagggcaatggtcacgccgcagccagtggtattccgggattagatgcacttgcaggtgttaagattgcacccgcccagttgggtgatatccagcagcgctatatgaaagacttttcggccttatggcaggctatggctgaaggaaaagctgaggcgacgggcccgttgcatgatcgccgcttcgccggcgacgcctggcgtacgaaccttccgtaccgctttgctgcggctttctacttactgaatgcgcgcgcgcttacagaactggcagacgccgtggaggcagatgcaaagacgcgtcagcgcattcgcttcgcgatctcgcagtgggtagacgccatgtcaccagcgaattttttagcgactaacccagaagcacaacgcttacttattgaaagtgggggtgagagtttgcgtgctggcgtccgcaatatgatggaagatttgacccgtgggaaaatctctcagacagacgaaagcgcctttgaggtggggcgtaacgtcgccgttaccgagggtgcagttgtctttgaaaatgagtattttcaattacttcaatacaagccgcttaccgacaaagtccatgcacgtcccctgctgatggtcccgccgtgtattaacaagtattatattttggacttacaaccggagagttcgctggtacgccacgttgtggaacagggacatactgtatttctggtctcctggcgcaatcctgacgcgagcatggctgggagtacatgggatgattatattgagcacgcggcaattcgtgctatcgaagtagcacgtgatatctcaggccaggacaagatcaacgtactggggttttgtgttggaggtaccatcgtatcaacagcattagcagtgttggcagcacgtggtgaacatcctgccgcctcggtcactctgttgaccacacttcttgatttcgccgatacgggcattctggacgttttcgtagacgagggtcatgttcagttgcgtgaggccacattaggcggtggggccggtgccccttgcgctttgttacgtggcctggaattggcgaacactttttcctttttacgccctaacgatcttgtgtggaactacgtcgtggataattatcttaagggaaatactccggttccgttcgatctgctgttttggaatggggacgccactaacttgccgggtccgtggtactgttggtaccttcgtcacacgtatttacaaaacgaattaaaagtaccgggaaaactgactgtgtgtggagtacccgtagaccttgcttctattgatgttccaacttacatttacggctcgcgtgaggaccatatcgtaccgtggaccgcagcctatgcttccactgcacttcttgctaacaaattgcgcttcgtgttgggagcatctggccacatcgctggggtaattaatccacctgcgaagaacaagcgctcgcactggacgaacgacgctttacccgagagcccgcaacagtggcttgcgggagcaatcgagcaccatggcagctggtggcctgactggactgcttggctggccggccaggcgggagctaaacgcgcggcgcccgcaaattacggaaacgctcgttaccgtgccatcgagccagccccaggtcgctacgtaaaagcgaaagcctga

SEQ ID NO: 3 - DNA, 786 bp, Clostridium acetobutylicum
  source 1..786: mol_type = genomic DNA
atggaactgaataatgtgatcctggaaaaagaaggcaaagtggctgtcgtcacgattaaccgtccaaaagccctgaatgctctgaattccgacaccctgaaagaaatggactatgttatcggtgaaatcgaaaatgacagcgaggtgctggccgttatcttgacgggtgccggtgagaaaagcttcgtcgcaggcgctgatattagcgagatgaaggagatgaataccattgaaggtcgcaaattcggtattctgggcaataaagtgttccgccgcctggagctgttggaaaagccggtcatcgcggcagttaatggtttcgcgctgggtggtggttgtgagatcgcgatgtcttgcgatattcgtatcgccagcagcaacgcccgttttggtcagccggaagtgggtttgggtatcacgccaggttttggtggtacccaacgtctgagccgtctggttggcatgggtatggcgaaacaactgatcttcacggctcaaaacatcaaggctgacgaagcactgcgtattggcctggtcaacaaggtcgtggaaccgagcgagctgatgaatacggcgaaggaaattgcaaataagatcgtgagcaacgcgccggtggctgttaaactgagcaagcaggcgatcaatcgtggtatgcagtgtgatatcgataccgccttggcatttgaaagcgaggcatttggtgaatgtttcagcacggaggatcagaaagacgcgatgacggcgtttatcgaaaaacgtaaaattgaaggtttcaaaaatcgttaa

SEQ ID NO: 4 - DNA, 849 bp, Clostridium acetobutylicum
  source 1..849: mol_type = genomic DNA
atgaaaaaagtttgcgtgatcggcgctggcactatgggcagcggtatcgcccaggcattcgcggccaaaggcttcgaagttgtcctgcgtgatatcaaagacgaatttgttgaccgtggcctggattttatcaacaaaaacctgagcaaactggttaaaaagggtaaaattgaagaagctacgaaagtcgaaatcctgactcgtatttctggcaccgtcgatctgaacatggccgcagattgcgatctggttattgaagctgccgttgaacgtatggacatcaaaaaacagatctttgctgatctggacaacatctgtaaaccagagaccatcctggctagcaacacctctagcctgtccatcaccgaagttgcttctgcgaccaaacgcccggataaagttatcggtatgcatttcttcaatccggctccggttatgaaactggtagaagttatccgcggcatcgctacgtctcaggagactttcgacgctgtgaaagagacctccatcgcaatcggtaaagatccagttgaagttgcagaagccccaggcttcgtggttaaccgtatcctgatcccgatgattaacgaagctgtgggtatcctggcagaaggtatcgcttctgttgaagatatcgacaaagcgatgaaactgggtgccaaccacccgatgggtccactggaactgggtgatttcattggtctggacatttgcctggcaatcatggacgttctgtacagcgaaacgggcgactctaagtaccgtccgcacactctgctgaaaaagtatgtgcgtgcgggttggctgggtcgtaaatctggtaaaggtttctacgactatagcaaataa

SEQ ID NO: 5 - DNA, 1629 bp, Ralstonia sp.
  source 1..1629: mol_type = genomic DNA
atgaaggtcattactgctcgcgaggccgcagcgttagttcaagacggttggactgtagcatcagcgggatttgtgggggctgggcacgccgaggctgttacagaggcacttgagcagcgcttcttgcagagcggccttcctcgcgacttgacgctggtttattccgccggccagggtgaccgcggtgcccgcggggtgaaccactttggcaatgccggtatgaccgcatccatcgtgggcggacactggcgttccgcaactcgtcttgccacgttagccatggcggaacagtgcgaagggtacaatcttccacaaggggtccttacgcatttataccgcgcgattgccggtggtaaaccgggagtgatgaccaagattggccttcatacttttgtagatccgcgcactgcacaagacgctcgctaccatggaggggctgtgaacgaacgtgcgcgtcaagcgattgcagaagggaaggcttgttgggttgacgctgtcgattttcgtggtgacgagtatttattctatccaagctttcccatccattgtgctttgattcgttgcaccgcagcagacgcacgcggcaatttatccactcatcgcgaagcgttccaccatgagttgttagcgatggcgcaagctgctcacaacagtgggggtatcgttattgcccaagtggaatcgctggttgaccaccatgaaatccttcaggcaatccatgtaccggggatcttggttgattacgtggtcgtctgtgacaaccctgcgaatcaccagatgacctttgcagagagttataatccagcctacgttactccatggcaaggtgaggcggcagttgctgaagcggaagctgctcccgtggcagcaggccctttggatgcccgtactatcgttcagcgtcgcgccgttatggagctggcccgtcgtgccccacgcgtcgtaaatttaggcgtggggatgccagcggctgtaggcatgttggcgcatcaagcagggctggacggattcactctgacagtcgaggccggaccgattggtggcaccccagcggatggattgtcgttcggtgcatcagcttacccagaagcagtggtagaccaaccagcccaattcgacttttatgagggtggaggaatcgacctggccatccttggtctggcggagcttgatggtcatggtaatgtaaacgtatcaaaatttggtgaaggggagggagcctcgattgcaggagtcggggggtttatcaacatcactcaatctgcacgtgcagtggtctttatgggtaccttaacggcgggtggcttagaggtccgtgccggggacgggggtttacaaattgtgcgcgaggggcgtgtgaagaagatcgtcccagaggtgagccacctttcgtttaacggaccatacgtcgcctcattgggtatcccggtattatatatcaccgaacgtgccgttttcgaaatgcgtgcgggggcggatggggaagcgcgtttgacgctggtagagattgcaccaggtgtggaccttcaacgtgatgtgctggaccaatgctcaacacctatcgcggtcgcacaagatttacgtgaaatggatgcgcgcctgttccaggctgggcccctgcacttataa

SEQ ID NO: 6 - DNA, 405 bp, Aeromonas caviae
  source 1..405: mol_type = genomic DNA
atgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaa

SEQ ID NO: 7 - DNA, 1527 bp, unidentified
  source 1..1527: mol_type = genomic DNA
atggcgaacgttctgaaaaccattcgttctggcgacgactatatcgaaagcctgcgtggtcgcgacctgaaggtttacctgttcggtgaactggtgaaagagcctgtagaccacccgatgatccgtccgagcatcaacgctgttgcggaaacctatgacctggctctgcgtgaagaagctctggcatctgcggattcctccattactggtctgaaagttaatcgttttctgcacatcgccgaatccgctgaagacctggttctgcagaacaaaatgcagcgcaaactgggccagaacactggcacttgctttcagcgttgcgtaggcatggacgcgatgaactctctgcactccaccaccttcgagattgatgagaaacacggtactgactaccacaaacgtttcctggagtttgtaaaaatggttcaacaggagaacctggttatcggtggcgctatgacggatcctaaaggcgaccgttccaaaggtccgagcgaacaagatgacccggatctgttcacccgtatcgtcgataccgacgagaagggcgtttatgtatccggtgccaaagcgcaccaaaccggctgcattaattcccactggatcatcctgatgccgaccattcgtctgaccgaatccgacaaagactgggccatcgtcggcgcaatcccagccgacgcaaagggtgtgacctacatctacggtcgtcagtcttgtgatactcgttctatggaagagggcgacattgatgatggcaacgctaaattcggtggtcaggaagctctgatcattctggatcgcgtattcatcccgtgggacaaagttttcatgcacggcgagtacgaattcgcttctatgctggtagaacgtttcacctgctaccatcgtcgtagctacgtctgtaaaactggtctgggcgatgttctgatcggtgcggctgctaccattgctgactacaacggtgtcccgaaggtatcccacatcaaggataaaatcatcgaaatgacccacctgaacgaaaccatttttgcggctggtatcgcgtcctcccatcagggtcagaaaatgaaatccggcgtatacctgaacgatgacatgctggcgcaggtatgcaaacataacgtgacccgtttcccgtacgaaatctcccgtctggcccaggatattgctggcggtctggtcgtaaccctgccgtccgagaaagattttcgccatccggaagctggcccactgctgaagaaatatctggctggccgtaaaggcgtggatgttgaaaaccgtatgcgtattctgcgtctgattgagaacatgaccctgggccgcaatgcagtaggttacctgacggagtctatgcacggtgctggttctccgcaagctcagcgtatccagatccagcgtcagatgcaggtaggctacaaaaaaaacctggctaaaaacctggcaggcatcaccaacgacgtagaggaaccgaaagaatcttctgaatattttaaacgcgtgttcaagaccaaagattccgtgctgtaa

SEQ ID NO: 8 - DNA, 1524 bp, unidentified
  source 1..1524: mol_type = genomic DNA
atgcctatcaagaatggcgcggaatacattcaatctttacgtgggcgtaaactgaaggtctatcttttcggagaactggtaaaggaaccagtagaccacccaatgatccgcccgagcatcaatgccgttgcggagacttacgacctggccgtcgaagagcctgagttagcatcggctaagagcagtctgtcaggtattcaagtcaaccgtttccttcatatcgctgagagtgcacaagatttagtgaatcaaaaccgtatgcaacgtaaactggggcagttaactggtacgtgtttccaacgttgcgttggcatggacgcgttgaactcactttatagtactaccttcgagattgatcagaagcatgggacccaataccacaagcgcttgattgagtttatcaaaatggtacagcgcgaaaatttggtgattggtggcgccatgacggacgttaaaggagatcgtagtctgtcgccttctcagcaggaggacccggatatgttcgtgcacattacgaagcgtgattcgaaaggtgtctatatcacaggcgccaaagctcaccaaaccggatgcattaactcacattggattatcgtgatgccgactatgcgcttgaaggccgaggacaaggactacgcaatcgtgggcgcgattccggttgatgccccaggtatcacttatatctacgggcgccaatcgtgcgatacacgttctatggagggaggtgagatcgatgccgggaacgtgatgtattccggtcaagaagctatggtgatctttaaagacgtgttcattccgaatgaattgattttcatggatggagagtacgaattcgcctcaatgttagttgaacgttttacctgctatcatcgtcgctcctacgtgtgtaaaaccggtctgggggacgttcttatcggagccgcagcggccatcgcggactataacggcgttgctaacgcgtctcatatcaaagataaattggtagaaatgacttatttgaacgaggcgatctttggagctgggatcgcgtccagttacatggcacataaaaccgcctccggcaactggcaaaatgacgacatgttggccaacgtctgcaagcactccgtgacccgctttccgtatgaaattggccgtatggcccaagacattgctggtggtttaatggtaactatgccctctgagaaagaatttcgctcgccggaaactggcccgctgctggagaagtatttaaagggccgtaaaggcgtaagcaccgaaaatcgcatgcgtatcctgcgcttaattgaaaatatgactttgggacgcaacgcagtggggtatcttactgagagtatgcacggggccggatcacctcaagcgcagcgtattcaaatcgcgcgtcaaatgcaattagaatacaagaaaaaagctgccaaaaaacttgcgaatgtgaaagagggtgaagctgctgagactgcgcctgaacaatctgattactttgacaagatcttcggtaccaaggataagaaaaaggtgaaatag

SEQ ID NO: 9 - DNA, 1521 bp, unidentified
  source 1..1521: mol_type = genomic DNA
atgcagaaaactgtcaaacccatccgtaccggggaggaatacattgaatctcttaaagggcgtgatctgaaagtatatttgtttggggaactggtgaaagaacctgtctcccaccctatgatccgcccctcaattaacgcggtcgctaaaacatacgaccttgctgttgaagaagaggagcttgcattcccaaaatctagtatttcaggtgaacgcgtcaatcgtttcctgcacatcgccgagtccgccgaggatctggttttgcagaacaagatgcaacgtaagctgggacagttaactggaacctgctttcaacgttgcgtggggatggatgccatgaactctctgcatagcacgacatttgaaatcgatgagaagcatggaacaaagtatcaccagcgccttttggagttcatcaaaatggtgcaacacgaaaacttagtaattggaggggccatgacggatgtcaaaggggaccgctctttggcgccgagcgaacaagaagatccggatttattcctgcatattgttgatcgcgacgataaaggagtttacgtcacaggggcgaaagcccaccagaccggaaccatcaattctcactggatgattgtaatgcctacaatgcgccttttagaaaatgacaaagattacgctattgttggtgcgctgcctgtcgatgctcctggaatcacttacatttatggccgtcagtcttgcgatacacgcagtatggagcctggtgatatcgatgtgggcaattcagagtttggcgggcaggaaactatggttatttttgaccgtgttttcattcctaacgagatgatttttatggacggagaatacgaatttgcctctatgctggtagagcgtttcacctgctatcaccgccgtagctacgtatgcaagactggattgggggacgtacttattggagccgccgctgcgattgcggagtacaatggagtaccaaaggtatcacacattaaggataagattattgagatgacccaccttaatgagtccatttacgcagcgggtatcgccgcgtcttatcaaggtcacaaaatgaaatctggggtttttctgaacgacgacatgctggcaaatgtatgcaaacataatgtaacgcgtctgccctatgagttagcccgtcttgctcaggatgtggccggaggcatcttggtcacactgccgtccgaggcggaatttcgctcagctgagacgggcccgttgttagagaaatacttgaagggtaaatctggcattgaagtcgaaaaccgcatgcgcattctgcgtctgatcgaaaacatgaccatgggccgcaatgcggtggggtatctgactgagagcatgcacggcgcgggaagcccccaagctcagcgcattcagatcgctcgtcagatgcagttgggatacaagaagcagttagctaaaaatcttgcgaaggtaaaagaagaccccgaagaaatgaaagagaactcggagtatttcaaacgcgtttttaagatccctgaaaagtaa

SEQ ID NO: 10 - DNA, 1527 bp, unidentified
  source 1..1527: mol_type = genomic DNA
atggcaaacgtactgaaaactatccgcaccgggaacgactacattgaatctcttcgtggtcgtgatttaaaagtttatttatttggggaacttgtgaaggagcctgttgaccatccgatgatccgccccagcatcaacgcagtcgctgagacatatgacttagccgtccgcgaggaggagcttgcatctgctcatagttcgctgacgggattgacggtcaaccgttttttacatattgcggaatctgcccaggatttggtcttgcagaataaaatgcaacgcaagttaggacaaaatacgggcacttgctttcaacgctgtgtcggaatggatgctttgaacgcactgcattcaacgacttttgagatcgacgaaaaatacaaaaccaactaccacaagcgctttttggaatttgttaaaatgatgcaaaaagaaaatcttgttatcggaggcgcgatgacagatcctaagggggatcgtagtaaaggtccagccgagcaggacgatcccgacctgttcacgcgtgtggtatcaaaagacgataaagggatctacgtttcgggagctaaggcacaccagacgggttgcatcaacagccactggattattttgatgccgaccgtacgcttaacggagaatgataaggattgggctgtcgtaggagcaatcccagcggacgctaagggcgtcacgtacatttatggccgtcagagctgcgacacccgctcgatggaagaaggcgacatcgatgacgggaatgccaaattcggtggtcaagaagcgttgatgatccttgagaacgtttttattccctgggataaggtctttatgtatggtgaatacgaattcgcttccatgttgattgaacgctttacctgttatcaccgccgcagttacgtttgtaagaccggtttaggggacgtgttgatcggagcggctgccgccatcgctgattacaatggcgtgccgaacgtctcccatattaaagataaaatcatcgaaatgacccatttaaatgaaacaatctttgccgctggtattgcgagctcccaccaggggcataaaatgaaaagcggggtttatttgaacgaggacatgctggctcaggtgtgtaaacacaatgtcactcgtttcccttatgagatttcccgcttagctcaagacattgcagggggcttggtggtaacgttgccctcagagaaagatttccgtcatcctatcgccggtcctatgttgaagaaataccttaaggggcgcaaaggcgccgacgtcgaaaaccgtatgcgtattctgcgtttaattgaaaatatgaccttggggcgcaacgccgtcgggtatttaacggaatccatgcacggagccgggtctcctcaggcacaacgtattcagatgcagcgtcagatgcagattggttacaaaaaaaatttggcgaaaaatctggcgggtatcacaaatgacattgagaagccaaacgaaccttccgattactttaaccgcgtgtttaagaccaaagattcggttttatag

SEQ ID NO: 11 - DNA, 1527 bp, unidentified
  source 1..1527: mol_type = genomic DNA
atgcaaaaaaccgtgcgccctattcgcacgggagaggagtacatcgagagtcttaaaggacgtaatctgaaggtgtaccttttcggcgaattggtgaaggagccggtcgatcaccccattatccgcccgtcaatcaatgcagtggcaaaaacttatgatctggctgttgaggaagaagacttagctagcgctaagtcatcaattacgggtgagcgcgtaaatcgttttcttcacattgcggagtctgctcaagacgtcgtattgcaaaataagatgcagcgcaagcttggtcagttgacgggtacgtgttttcagcgttgcgttggcatggacgcgctgaattctttgcattcgacaaccttcgagatggacgagaaacacggtacaaaataccaccagcgtcttttagagttcattaaaattgttcagcacgaaaatttggtcatcggcggggcgatgactgacgtcaagggcgaccgctccaagggaccctctgagcaggaagatcctgacctgttcttacgtattgttgaacgcgacgacaagggagtatatatcagcggtgctaaagctcaccagaccggttgtgtaaattcccattggatgatcgttatgccagcggtacgcttgaccgaggctgacaaggactatgctgttgtaggggcaattcccgttgatgctcccggcattacctacatttacggccgccagtcgtgcgacactcgctcgatggaacctggggacatggatcagggcaactcccaattttctgggcaggaagcaatggtaatctttgataaagcgttcattccaaatgaattcattttcatggatggagagtatgaattcgcatctatgttgattgaacgcttcacttgctatcatcgccgctcatacgtctgtaagaccggggtaggagacgttttgatcggagcagcggcgagcattgctgattataacggagttcccaaggtctcccacattaaagataaacttgttgaaatgactcatcttaatgaaacgatttatggtacgggcatcgcgagctcctatcaatcgcagaagatgaagtcaggcgtatggcaaaacgacgagatgctggctaacgtttgtaagcacaatgtaacgcgctttccgtaccagatcggccgcttcgcccaagacattgcaggggggttgatggtgactctgccctctgaagctgagtttcgtagccctgaaacgggtcctttattaaaaaagtatttaaagggacgtaaaggcgttgacgttgagaacaaaatgcgtattttacgccttatcgagaatatgacgatgggtcgcaacgccgtcggctatttgaccgaatctatgcatggcgccggtagtccccaagcccaacgcattcaaattgcgcgccaaatgcaattggggtataaaaaaaaacttgctaaaaacctggcaaaagtccaggaagagggggaaaaaaccctggagaatagtgattacttcaaccgtgtattcaaactggacgactctaaagagtaa

SEQ ID NO: 12 - DNA, 115 bp, synthetic construct
  source 1..115: mol_type = other DNA
gagggcggggttttttttcttctagattgacagctagctcagtcctaggtataatgctagcgtgtttgcctgcccaacagaggaggacgccgcatgagcgcacaatccctggaag

SEQ ID NO: 13 - DNA, 41 bp, synthetic construct
  source 1..41: mol_type = other DNA
cgaattcctgcagcccggggttaaggcagcttgaccacggc

SEQ ID NO: 14 - DNA, 42 bp, synthetic construct
  source 1..42: mol_type = other DNA
ccccgggctgcaggaattcgatatgaaggtcattactgctcg

SEQ ID NO: 15 - DNA, 41 bp, synthetic construct
  source 1..41: mol_type = other DNA
cgaggtcgacggtatcgatattataagtgcaggggcccagc

SEQ ID NO: 16 - DNA, 189 bp, synthetic construct
  source 1..189: mol_type = other DNA
gcttatcgataccgtcgacctcgccagtctggcctgaacatgatataaaatgaatataaatagacagaatagggttatttatggatttatatcactttacagcctgtctcttgatcagatctggccgcctaggccgaattcgagctcggtaccaattcaggaggtttttatggcgacgggaaaaggtgc

SEQ ID NO: 17 - DNA, 51 bp, synthetic construct
  source 1..51: mol_type = other DNA
caaaagctgggtaccgggccccccctcgatcaggctttcgcttttacgtag

SEQ ID NO: 18 - DNA, 113 bp, synthetic construct
  source 1..113: mol_type = other DNA
gagggcggggttttttttcttctagattgacagctagctcagtcctaggtataatgctagcgtgtttgcctgcccaacagaggaggacgccgcatggcgaacgttctgaaaac

SEQ ID NO: 19 - DNA, 40 bp, synthetic construct
  source 1..40: mol_type = other DNA
cgaattcctgcagcccggggttacagcacggaatctttgg

SEQ ID NO: 20 - DNA, 39 bp, synthetic construct
  source 1..39: mol_type = other DNA
gtgcacgctagcttaaaacgtcgccagtctggcctgaac

SEQ ID NO: 21 - DNA, 42 bp, synthetic construct
  source 1..42: mol_type = other DNA
ctcactatagggcgaattggtcaggctttcgcttttacgtag

SEQ ID NO: 22 - DNA, 73 bp, synthetic construct
  source 1..73: mol_type = other DNA
acagaaggcttaaggatcctcggtttcttcagagatttaaaaccactatcaatatattcatgtcgaaaatttg

SEQ ID NO: 23 - DNA, 42 bp, synthetic construct
  source 1..42: mol_type = other DNA
tgcatgcctgcaggtcgactttatttgcgctcaactgctaac

SEQ ID NO: 24 - DNA, 106 bp, synthetic construct
  source 1..106: mol_type = other DNA
gagggcggggttttttttcttttgacagctagctcagtcctaggtataatgctagcttaattaatctagggtaccaggaggtttttatgcctatcaagaatggcgc

SEQ ID NO: 25 - DNA, 47 bp, synthetic construct
  source 1..47: mol_type = other DNA
gttccataaaaacctcctcccctatttcacctttttcttatccttgg

SEQ ID NO: 26 - DNA, 106 bp, synthetic construct
  source 1..106: mol_type = other DNA
gagggcggggttttttttcttttgacagctagctcagtcctaggtataatgctagcttaattaatctagggtaccaggaggtttttatgcagaaaactgtcaaacc

SEQ ID NO: 27 - DNA, 45 bp, synthetic construct
  source 1..45: mol_type = other DNA
gttccataaaaacctcctcccttacttttcagggatcttaaaaac

SEQ ID NO: 28 - DNA, 106 bp, synthetic construct
  source 1..106: mol_type = other DNA
gagggcggggttttttttcttttgacagctagctcagtcctaggtataatgctagcttaattaatctagggtaccaggaggtttttatggcaaacgtactgaaaac

SEQ ID NO: 29 - DNA, 43 bp, synthetic construct
  source 1..43: mol_type = other DNA
gttccataaaaacctcctcccctataaaaccgaatctttggtc

SEQ ID NO: 30 - DNA, 106 bp, synthetic construct
  source 1..106: mol_type = other DNA
gagggcggggttttttttcttttgacagctagctcagtcctaggtataatgctagcttaattaatctagggtaccaggaggtttttatgcaaaaaaccgtgcgccc

SEQ ID NO: 31 - DNA, 42 bp, synthetic construct
  source 1..42: mol_type = other DNA
gttccataaaaacctcctcccttactctttagagtcgtccag

SEQ ID NO: 32 - DNA, 47 bp, synthetic construct
  source 1..47: mol_type = other DNA
gagggcggggttttttttcttctagtcgccagtctggcctgaacatg

SEQ ID NO: 33 - DNA, 41 bp, synthetic construct
  source 1..41: mol_type = other DNA
ttaaggcagcttgaccacgggttccataaaaacctcctccc

SEQ ID NO: 34 - DNA, 71 bp, synthetic construct
  source 1..71: mol_type = other DNA
agtcgacctgcaggcatgcatcgccagtctggcctgaacatgatataaaatgtgtttgcctgcccaacaga

SEQ ID NO: 35 - DNA, 40 bp, synthetic construct
  source 1..40: mol_type = other DNA
ctatgaccatgattacgccattaaggcagcttgaccacgg

SEQ ID NO: 36 - DNA, 40 bp, synthetic construct
  source 1..40: mol_type = other DNA
agggcggggttttttttcttccagcctgtttacgatgatc

SEQ ID NO: 37 - DNA, 43 bp, synthetic construct
  source 1..43: mol_type = other DNA
ctggtaccctagattaattaatccttaactgtatgaaattggg

SEQ ID NO: 38 - DNA, 42 bp, synthetic construct
  source 1..42: mol_type = other DNA
ctaggtataatgctagcttaatccagcctgtttacgatgatc

SEQ ID NO: 39 - DNA, 43 bp, synthetic construct
  source 1..43: mol_type = other DNA
ctcctggtaccctagattaattccttaactgtatgaaattggg

SEQ ID NO: 40 - DNA, 49 bp, synthetic construct
  source 1..49: mol_type = other DNA
gttacgtgaaaggaacaaccaagtcgatcgccagtctggcctgaacatg

SEQ ID NO: 41 - DNA, 43 bp, synthetic construct
  source 1..43: mol_type = other DNA
taagcgtgagagttaaaaaaaagttaaggcagcttgaccacgg